(19) United States
(12) Patent Application Publication
(10) Pub. No.: US 2016/0187341 A1
(43) Pub. Date: Jun. 30, 2016

(54) KERATINS AS BIOMARKERS FOR CERVICAL CANCER AND SURVIVAL

(71) Applicant: THE RESEARCH FOUNDATION FOR THE STATE UNIVERSITY OF NEW YORK, Albany, NY (US)

(72) Inventors: Kenneth R. SHROYER, Setauket, NY (US); Luisa F. ESCOBAR-HOYOS, Calverton, NY (US); Emily I. CHEN, Stony Brook, NY (US)

(73) Assignee: THE RESEARCH FOUNDATION FOR THE STATE UNIVERSITY OF NEW YORK, Albany, NY (US)

(21) Appl. No.: 14/910,785

(22) PCT Filed: Aug. 8, 2014
(86) PCT No.: PCT/US2014/050267

§ 371 Date: Feb. 8, 2016

Related U.S. Application Data
(60) Provisional application No. 61/865,750, filed on Aug. 14, 2013.
(60) Provisional application No. 61/863,671, filed on Aug. 8, 2013.

(51) Int. Cl. G01N 33/574; C12Q 1/68

(52) U.S. Cl. CPC G01N 33/57411; C12Q 1/6886; C12Q 2600/158; G01N 2333/4742

(57) ABSTRACT

The current disclosure provides methods for detecting and analyzing KRT4 and KRT17 expression in a sample obtained from a test subject. The current disclosure pertains to methods and kits for identifying a mammalian subject with cervical cancer or non-cancerous lesions of the cervix. The current disclosure further provides methods and kits for determining the likelihood of survival or treatment outcome of a subject having cervical cancer by determining the expression level of KRT17 in a sample.

This application claims benefit of U.S. Provisional Application No. 61/863,671, filed Aug. 8, 2013, and U.S. Provisional Application No. 61/865,750, filed Aug. 14, 2013, the entire contents of which are incorporated herein by reference.

### STATEMENT REGARDING FEDERALLY SPONSORED RESEARCH

The present disclosure was made with government support under grant numbers AI091175 and CA140084 awarded by the National Institutes of Health. The government has certain rights in the disclosure.

### FIELD OF THE DISCLOSURE

The current disclosure relates to a method of diagnosing abnormalities of the cervix, which indicate the presence of cervical cancer or the presence of a pre-cancerous lesion in a subject. The current disclosure further provides methods of analyzing the protein expression levels of Keratin 4 and Keratin 17 in subjects in order to determine the presence of cervical cancer or the presence of a pre-cancerous lesion in a subject. The current disclosure further relates to methods for analyzing Keratin 17 in subjects in order to predict patient prognosis and survival.

### BACKGROUND

Cervical cancer is the second leading cause of death among women worldwide, but is a less common cause of cancer mortality in most industrialized nations, due largely to the success of cervical cancer screening cytology (i.e., the “Pap test”). In the United States, 12,200 new diagnoses and 4,200 cancer deaths were reported in 2012. See Siegel R, et al., CA: A Cancer Journal for Clinicians. 2012; 62: 10-29. In addition, three million cervical cytology specimens have abnormal cytologic findings that require further evaluation by colposcopy. See Schiffman M, et al., JNCI. 2011; 103: 368-83. Although high-risk human papilloma virus (HPV) testing is widely used to improve the accuracy of cervical cancer screening, positive test results have poor specificity for underlying high-grade squamous intraepithelial lesion (HSIL) or squamous cell carcinoma in patients with a cytologic diagnosis of atypical squamous cells of undetermined significance (ASC-US) or low-grade squamous intraepithelial lesion (LSIL) because most HPV infections are transient and are unlikely to result in malignant transformation. See Wright T C J. J Fam Pract. 2009; 58: S3-7. The histologic classification of HSIL can also be problematic, due to a variety of technical issues (e.g., specificity of staining) or diagnostic challenges (e.g., lack of a distinct biomarker) that contribute to both false negative or false positive diagnoses. While p16^{INK4a}/Ki-67 dual stain approaches and other biomarkers may provide an objective basis to support the histologic diagnosis of HSIL and squamous cell carcinoma, most are expressed in a high proportion of LSILs. See, for example, Samarawardana P, et al., Appl. Immunohistochem. Mol. Morphol. 2011; 19: 514-8; Yamazaki T, et al., Pathobiology. 2006; 73: 176-82; and Masoudi H, et al., Histopathology. 2006; 49: 542-5.

Therefore, there remains an important clinical need to: (i) identify new cervical cancer biomarkers that could improve specificity for the detection of HSIL/squamous cell carcinoma versus normal/LSIL in tissue biopsies; (ii) to focus resources on treatment of patients that are most likely to benefit from colposcopy and subsequent treatment intervention; (iii) and avoid overtreatment of patients who are likely to have only transient HPV infections. See Narayan K. Int. J. Gynecol. Cancer. 2005; 15: 573-82. Furthermore, the validation of prognostic markers in squamous cell carcinoma patients could improve their clinical management and treatment outcome. For example, in clinical practice most squamous cell carcinoma patients undergo radical hysterectomy and may also undergo post-operative chemotherapy and radiotherapy based on the tumor stage. However, treatment outcomes of these patients vary significantly. See, e.g., Schwarz J K, et al., JAMA. 2007; 298: 2289-95; and Eifel P J, et al., J. Clin. Oncol. 2004; 22: 872-80.

In view of the deficiencies above, the current disclosure identifies and validates biomarkers for HSIL and squamous cell carcinoma including, for example, keratin 4 (KRT4) and keratin 17 (KRT17), and further characterizes KRT17 as a prognostic biomarker for patients with cervical squamous cell carcinoma.

### SUMMARY OF THE DISCLOSURE

The current disclosure shows that keratin 4 (KRT4) and keratin 17 (KRT17) are predictive biomarkers for diagnosing cervical cancer and diagnosing abnormalities of the cervix that indicate the presence of cervical cancer or the presence of a pre-cancerous lesion in a subject.

In one aspect of the current disclosure KRT4 is validated as a clinical biomarker for the diagnosis of squamous cell carcinoma of the cervix and high-grade squamous intraepithelial lesions (HSIL). In certain embodiments, the expression of KRT4 is reduced in subjects with squamous cell carincoma of the cervix and HSIL, when compared to that of normal control samples, a reference sample, and/or low-grade squamous intraepithelial lesions (LSIL).

In another aspect of the present disclosure, KRT17 is identified as a clinical biomarker for the diagnosis of a subject having or that may have squamous cell carcinoma of the cervix. In certain embodiments, KRT17 expression levels were significantly increased in subjects with squamous cell carcinoma of the cervix or HSIL, when compared to that of normal control samples or reference samples, and/or low-grade squamous intraepithelial lesions (LSIL). In another embodiment, KRT17 expression was absent or detected at negligible levels in normal squamous mucosa or subjects characterized as having LSIL, which indicates the absence of squamous cell carcinoma of the cervix or a pre-cancerous lesion thereof in such subject.

Taken together, the current disclosure reveals that the loss or reduction of KRT4 expression and/or increase of KRT17 expression is a critical event in the development of cervical cancer. A discovery that can be incorporated in the present methods for identifying a subject having cervical cancer or a pre-cancerous lesion thereof.

In one aspect of the present disclosure, significant increases in KRT17 expression levels have been observed in squamous cell cancer samples relative to non-cancerous control samples or LSIL samples, which have been correlated with a reduced incidence of survival and/or a negative treatment outcome. Hence, in certain embodiments of the instant disclosure when an increased level of KRT17 expression is detected in a sample obtained from a subject, the subject is likely to have a reduced likelihood of survival and/or negative treatment outcome when compared to a subject diagnosed with cervical cancer that does not have an increase in KRT17 expression over that of normal squamous mucosa or a control sample.

### BRIEF DESCRIPTION OF THE DRAWINGS

FIG. 1: Experimental design for mass spectrometry-based biomarker discovery and immunohistochemical-based biomarker validation. A. Tissue microarrays designed for each diagnostic category. Specifically, normal: non-cancerous ectocervical squamous mucosa, LSIL: low-grade squamous intraepithelial lesion, HSIL: high-grade squamous intraepithelial lesion, SCC: squamous cell carcinoma. B. Subcellular localization of proteins identified from formalin-fixed paraffin-embedded archived cervical tissues based on the Gene Ontology classification. Protein percentages for each subcellular category are shown.

FIG. 2: Detection of Keratin 4 expression in squamous cell carcinoma. A. Keratin 4 (KRT4) immunohistochemical staining in representative cases. Normal: non-cancerous ectocervical squamous mucosa, LSIL: low-grade squamous intraepithelial lesion, HSIL: high-grade squamous intraepithelial lesion, SCC: squamous cell carcinoma. The scale bar represents 50 μm. B. Expression data of KRT4 in each diagnostic category based on the PathSQ immunohistochemical scores, which is based on the percentage of positive cells with strong staining (n=25-27 cases per diagnostic category). Mean value (bold dashed line) and median (solid line). * p>0.001 by Kruskal-Wallis and Wilcoxon rank-sum test.

FIG. 3: Detection of Keratin 17 in high-grade squamous intraepithelial lesion and squamous cell carcinoma. Normal: non-cancerous ectocervical squamous mucosa, LSIL: low-grade squamous intraepithelial lesion, HSIL: high-grade squamous intraepithelial lesion, SCC: squamous cell carcinoma. A. Keratin 17 (KRT17) immunohistochemical staining in representative cases from each diagnostic category. The scale bar represents 50 μm. B. Expression data of KRT17 in each diagnostic category based on the PathSQ immunohistochemical scores, determined by the percentage of positive cells exhibiting strong staining (n=25-27 cases per diagnostic category). Mean value (bold dashed line) and median (solid line). * p>0.05 by Kruskal-Wallis and Wilcoxon rank-sum test.

FIG. 4: Correlation of Keratin 17 expression with non-cancerous pathologies. A. No statistically significant change in KRT17 expression was observed in samples obtained from subjects having: immature squamous metaplasia, mature squamous metaplasia, inflammation (cervicitis), wound-healing (biopsy site changes), or herpes simplex viral infection. Mean value (bold dashed line) and median (solid line). * p>0.001 by Kruskal-Wallis. B. KRT17 expression was detected in immature squamous metaplasia (Left), mature squamous metaplasia (Right) and endocervical reserve cells (Bottom). Twelve out of seventeen endocervical mucosal reserve cell samples stained positive for KRT17. Scale bar represents 20 μm. C. Correlation between keratin 17 expression and high-risk HPV type in squamous cell carcinomas (SCC). (Left) High-risk HPV type percentages in squamous cell carcinoma cases (n=25). 54% and 28% of samples were positive for HPV type 16 or 18, respectively. Four samples revealed a dual HPV infection, including HPV16 and other high-risk HPV. One case had HPV39 alone. High-risk HPV typing was performed by multiplex PCR and capillary electrophoresis. (Right) Box plots of KRT17 PathSQ immunohistochemical quantification in squamous cell carcinomas (n=25). Mean value (bold dashed line) and median (solid line). No statistical significant differences were detected (p>0.05) by the Kruskal-Wallis test.

FIG. 5: Kaplan-Meier curves of the overall survival of patients diagnosed with squamous cell carcinoma with high or low KRT17 (K17) expression. A. Results are shown for 65 squamous cell carcinoma cases with high-KRT17 versus low-KRT17 ImageJ scores, showing a higher probability of patient survival beyond 5 years (60 months) and 10 years (120 months) for when patients exhibit low-KRT17 expression. B. Results are shown for 65 squamous cell carcinoma cases with high-KRT17 versus low-KRT17 PathSQ scores revealing a higher probability of patient survival beyond 5 years (60 months) and 10 years (120 months) for when patients exhibit low KRT17 expression. C. Immunohistochemical staining of KRT17 in representative squamous cell carcinoma cases with low (left) or high (right) KRT17 expression. Images were taken at 20× magnification. The scale bar represents 100 μm.

FIG. 6: Correlation of Keratin 17 expression with cancer stage, grade, lymph node status, and primary versus metastatic tissue site. Box plot of KRT17 PathSQ immunohistochemical quantification in squamous cell carcinomas (n=65). A. Evaluation KRT17 expression in different stages of cancer. T1: cervical carcinoma confined to the uterus, T2: tumor invades beyond the uterus but not to pelvic wall or to lower third of the vagina (n=4), T3: tumor extends to the pelvic wall and/or involves the lower third of the vagina and/or causes hydronephrosis or nonfunctioning kidney (n=18). AJCC staging (16). B. Evaluation of KRT17 expression in different histological grades of cancer. G1: well differentiated (low grade); G2: moderately differentiated; G3: poorly differentiated. C. Evaluation of KRT17 expression in cancers with various lymph node status. NO: node negative; Ni: regional (pelvic) node metastasis. Nine cases were not assessed. D. Evaluation of KRT17 expression in matched primary and metastatic tumors from same subject. Mean value (bold dashed line) and median (solid line). No statistically significant differences were detected (p>0.05) by Wilcoxon rank-sum test.

FIG. 7: Validation of KRT17 as a prognostic indicator of patient outcome in cervical cancer, independent of tumor stage. A. Representative hematoxylin and eosin (H&E) and immunohistochemical (IHC) stains for keratin 17 (K17) in squamous cell carcinomas of the cervix, with low and high K17 expression. Both representative samples are the same stage and tumor grade. Scale bar, 100 μm. B-E. IHC scoring by PathSQ method on high and low K17 samples (B), and relative expression of keratin 17 (KRT17) mRNA levels from dissected formalin-fixed paraffin embedded squamous cell carcinomas (C). IHC scoring by PathSQ method by tumor stages (D); T1+T2: cancer is confined to the cervix, while T3+T4 represents cancer that extends beyond the cervix. E. IHC scoring by Path SQ method by tumor grades. Grade G1 is a well differentiated tumor; G2: moderately differentiated; and G3 represents a poorly differentiated tumor. The horizontal dashed lines in the box plots represent the mean, while solid lines represent the median. Boxes represent the interquartile range, and the whiskers represent the 2.5^{th} and the 97.5^{th} percentiles. Black circles represent outlier samples from Mann-Whitney U tests. *** p<0.001. F-H. Kaplan-Meier curves depicting the probability of overall survival of cervical cancer patients (squamous cell carcinomas) stratified by K17 IHC status in primary tumors, low (≦50 PathSQ score) or high (≧50 PathSQ score) K17. All cases (F) and within stages T1+T2: cancer is confined to the cervix (G), while T3+T4 represents cancer that extends beyond the cervix (H). p-values were calculated using the log-rank test. I. The failure hazard for cervical cancer patients stratified by K17 status using a Cox proportional hazards model. J. Relative endogenous expression of K17 in cervical cancer cell lines, e.g., siHa, Caski, C-33A, HT-3, ME-180, and HeLa.

FIG. 8: Keratin 17 knockdown induces cell cycle arrest and decreased cell size. A. Cell proliferation of SiHa and CaSki cells after transfection with negative control siRNA or siRNA against KRT17 was determined by colorimetric method and analysis. G1-phase cell population in SiHa and CaSki cells with KRT17 knockdown by siRNA (B) or shRNA (E) compared to KRT17 expression using negative control siRNA or shRNA. C-D. Post-mitotic G1A-cell population (C) and KRT17 RNA quantification (D) in SiHa and CaSki cells with KRT17 knockdown by siRNA against KRT17, compared to negative control siRNA. F. Cell size measurement as determined by forward scatter (FSC) by flow cytometry analysis in SiHa and CaSki cells with KRT17 knockdown by shRNA compared to negative control shRNA. G. Quantification of senescence-associated P3-galactosidase in SiHa and CaSki cells with KRT17 knockdown by shRNA compared to negative control shRNA. H. G1-phase cell population in C-33A cells (i.e., cells devoid of endogenous KRT17) after transfection with human KRT17.

FIG. 9: Keratin 17 knockdown correlates with nuclear p27^{KIP1} accumulation. A-C. Representative western blots (A) and relative expression quantification (B-C) of p27^{KIP1}, phospho-pRb, p130 and cyclin A in SiHa and CaSki cells transfected with negative control siRNA or siRNA against KRT17. D. Quantification of nuclear p27^{KIP1} positive cells after immunofluorescent staining in cells transfected with negative control siRNA or siRNA against KRT17. E-F. Representative western blot (E) and relative expression quantification (F) of p27^{KIP1} in cytosolic (top) and nuclear (bottom) cellular fractions obtained from SiHa and CaSki cells stably transfected with negative control shRNA or shRNA against KRT17. G. Representative western blot detection of phospho-p27^{KIP1} using phospho-Histone H3 (Ser 10) antibody (p-p27^{KIP1} Ser10), and CDK2 in SiHa and CaSki cells transfected with negative control shRNA or shRNA against KRT17. H. Relative expression of p27^{KIP1} (CDKN1B) mRNA levels in cells transfected with negative control shRNA or shRNA against KRT17. I. Relative-gene expression of cyclin dependent kinase inhibitors by RT-quantitative PCR (RT-qPCR) for SiHa and CaSki cells transfected with negative control shRNA or shRNA against KRT17. J. Representative western blot detection of p21^{CIP1/WAF1} and p53 expression in CaSki cells transfected with negative control shRNA or shRNA against KRT17. Quantitative data are presented as averages±standard deviation. Statistical analyses were carried out by T-test or Mann-Whitney U. * p<0.05, ** p<0.01 and *** p<0.001.

Table 1: Demographic and clinical characteristics of cases. ^{a} Low-grade squamous intraepithelial lesion, ^{b} High-grade squamous intraepithelial lesion, c Squamous cell carcinoma, and d Clinical staging of tumors according to The AJCC cancer staging manual and the Annals of surgical oncology 17(6), 1471-1474.

Table 2: Keratin 4 and 17 receiver operating curves curve analysis and misclassification rate results between different diagnostic categories according to PathSQ score. ^{a} area under the curve, ^{b} confidence interval, ^{c} positive predictive value, ^{d} negative predictive value, ^{e} squamous cell carcinoma, ^{f} high-grade squamous intraepithelial lesion, ^{g} low-grade squamous intraepithelial lesion.

### DETAILED DESCRIPTION OF THE DISCLOSURE

To date, diagnostic markers (e.g., immunohistochemical markers) of cervical high-grade squamous intraepithelial lesion (HSIL) and squamous cell carcinoma (SCC) marginally improve diagnostic accuracy, and have no prognostic value. Conversely, the current disclosure identifies, characterizes and validates two novel biomarkers, i.e., KRT4 and KRT17, which improve diagnostic and prognostic accuracy for cervical HSIL and squamous cell carcinoma.

### Diagnostic Methods

One aspect of the present disclosure describes methods for using keratin 4 (KRT4) and/or keratin 17 (KRT17 or K17) as biomarkers of cervical high-grade squamous intraepithelial lesion (HSIL) and squamous cell carcinoma (SCC). Herein, KRT4 and KRT17 were identified from microdissected tissue sections obtained from formalin-fixed paraffin-embedded samples for each diagnostic category (i.e., non-cancerous ectocervical squamous mucosa, low-grade squamous intraepithelial lesion (LSIL), HSIL and SCC) and evaluated by mass spectrometry-based shotgun proteomics. The data revealed that KRT4 and KRT17 exhibited at least a two-fold difference in expression across diagnostic categories of SCC, and had a protein expression profile indicative of disease progression. Therefore, the instant disclosure shows that KRT4 and/or KRT17 expression can be measured as an indicator of the progression of non-cancerous squamous mucosa to SCC. For example, KRT17 expression is increased from normal tissue to LSIL, LSIL to HSIL, and HSIL to squamous cell carcinoma. In another example, KRT4 expression is decreased during the progression normal tissue to squamous cell carcinoma.

In view of the foregoing, KRT4 and KRT17 were selected for further validation as diagnostic biomarkers by immunohistochemical analysis of tissue microarrays. These immunohistochemical studies clearly show that KRT17 expression was significantly increased in HSIL and squamous cell carcinoma compared to normal ectocervical squamous mucosa and LSIL. Similarly, the immunohistochemical studies provided herein confirm that KRT4 expression was significantly decreased in squamous cell carcinoma compared to the other diagnostic categories (i.e., non-cancerous ectocervical squamous mucosa, low-grade squamous intraepithelial lesion (LSIL), HSIL).

One embodiment of the present disclosure provides a method for diagnosing a subject with squamous cell carcinoma, which includes obtaining a sample from a subject, and detecting the level of KRT17 expression in the sample. Whereby an increased level of KRT17 expression in the sample identifies the subject as having squamous cell carcinoma of the cervix.

In yet another embodiment of the present disclosure, KRT4 expression is measured as an indicator of the progression of non-cancerous squamous mucosa to SCC. Therefore, one embodiment of the present disclosure provides a method for diagnosing a subject with squamous cell carcinoma, which includes obtaining a sample from a subject, and detecting the level of KRT4 expression in the sample. Whereby a reduced level of KRT17 expression in the sample identifies the subject as having squamous cell carcinoma of the cervix.

In certain embodiments, a biological sample is obtained from the subject in question. A biological sample, which can be used in accordance with the present methods, may be collected by a variety of means known to those of ordinary skill in the art. Non-limiting examples of sample collection techniques for use in the current methods include; fine needle aspiration, surgical excision, endoscopic biopsy, excisional biopsy, incisional biopsy, fine needle biopsy, punch biopsy, shave biopsy and skin biopsy. Additionally, KRT4 and/or KRT17 expression levels can be detected from cancer or tumor tissue or from other body fluid samples such as whole blood (or the plasma or serum fractions thereof) or lymphatic tissue. In certain embodiments, the sample obtained from a subject is used directly without any preliminary treatments or processing, such as formalin-fixation, flash freezing, or paraffin-embedding. In a specific embodiment, a biological sample can be obtained from a subject and processed by formalin treatment and embedding the formalin-fixed sample in paraffin. In certain embodiments, a sample may be stored prior to use.

After a suitable biological sample is obtained, the level of KRT4 and/or KRT17 expression in the sample can be determined using various techniques known by those of ordinary skill in the art. In certain embodiments of the current disclosure KRT17 expression levels may be measured by a process selected from: immunohistochemistry (IHC), q-RT-PCR, northern blotting, western blotting, enzyme-linked immunosorbent assay (ELISA), microarray analysis, or RT-PCR.

In a specific embodiment, immunohistochemical analysis of KRT4 and/or KRT17 is conducted on formalin-fixed, paraffin-embedded samples. Here, normal cervical mucosa, LSIL, HSIL and squamous cell carcinoma from hematoxylin and eosin stained tissue sections are dissected by laser capture microscopy, collecting cells from each diagnostic category (i.e., non-cancerous ectocervical squamous mucosa, LSIL, HSIL, and SCC). Formalin-fixed, paraffin-embedded tissues are then incubated in 50 mM Ammonium Bicarbonate with protease cocktails to facilitate the reverse of protein cross-linking. The samples can then be further processed by homogenization in urea. The protein concentration can then be determined by any suitable method known to one of ordinary skill in the art.

In a specific embodiment, KRT4 and/or KRT17 protein detection is carried out via tissue microarray. For example, tissue containing normal cervical mucosa, LSIL, HSIL or squamous cell carcinoma can be obtained from paraffin blocks and placed into tissue microarray blocks. In certain embodiments, other sources of tissue samples can be used as control samples including, but not limited to, commercial tissue microarray samples, such as those obtained from HISTO-Array™. Tissue microarray slides for use in the current methods can then be processed, i.e., deparaffinized in xylene and rehydrated using an alcohol.

In certain embodiments, samples can be further processed by: incubation with a citrate buffer, applying hydrogen peroxide to block endogenous peroxidase, or by treating the sample with serum to block non-specific binding (e.g., bovine, human, donkey or horse serum). The samples are further incubated with primary antibodies against KRT4 and/or KRT17. Any antibody can be used against the KRT4 or KRT17 antigen including, but not limited to, mouse monoclonal-[E3] anti-human KRT17 antibody, mouse monoclonal-[6B10] anti-human KRT4 antibody, polyclonal antibodies against human KRT4 or KRT17, a monoclonal antibody or polyclonal antibody against a mammalian KRT4 or KRT17 protein domain or epitope thereof. In certain embodiments, after incubation with the primary antibody, samples are processed by an indirect avidin-biotin-based immunoperoxidase method using biotinylated secondary antibodies, developed, and counter-stained with hematoxylin. Slides can then be analyzed for KRT4 and/or KRT17 expression.

In certain embodiments, keratin expression is quantified by PathSQ method, a manual semi-quantitative scoring system, which quantifies the percentage of strongly stained cells, blinded to corresponding clinical data. In yet another embodiment, slides can be scored by the National Institutes of Health ImageJ 1.46, Java-based image processor software using the DAB-Hematoxylin (DAB-H) color deconvolution plugin. See Schneider C A, et al., Nat methods. (2012) 9:671-5 and/or by a manual semi-quantitative scoring system, which quantifies the percentage of strong-positively stained cells blinded to corresponding clinical data (PathSQ).

In yet another embodiment KRT4 and/or KRT17 expression can be determined using reverse transcriptase PCR (RT-PCR) or quantitative-RT-PCR. More specifically, total RNA can be extracted from a sample by using a Trizol reagent. Reverse transcriptase-PCR can then be performed using methods know by one of ordinary skill in the art. For example, 1 μg of RNA can be used as a template for cDNA synthesis and cDNA templates can then be mixed with gene-specific primers (i.e., forward, 5′-3′ primer sequence and reverse 3′-5′ sequence) for KRT17 or KRT4. Probe sequences for detection can also be added (e.g., Taqman or SYBR Green. Real-time quantitative PCR can then be carried out on each sample and the data obtained can be normalized to control levels of KRT4 or KRT17 expression levels as set forth in a control or normal sample. See, for example, Schmittgen, and Livak, Nature protocols (2008) 3: 1101-1108.

In one embodiment of the current disclosure, the amount of KRT4 and/or KRT17 in a sample is compared to either a standard amount of KRT4 and/or KRT17 present in a normal cell or a non-cancerous cell, or to the amount of KRT4 and/or KRT17 in a control sample. The comparison can be done by any method known to a skilled artisan. In a specific embodiment, the amount of KRT17 expression indicative of a subject having SCC includes, but is not limited to, a 5-10%, 10-20% increase over that of a control sample, or at least 20%, 30%, 40%, 50%, 60%, 70%, 80%, 90%, 100%, 200% or greater increase over that of a control sample, or at least a 0.25 fold, 0.5 fold, 1 fold, 1.5 fold, 2 fold, 3 fold, 4 fold, 5 fold, 10 fold, 11 fold or greater, increase relative to the amount of KRT17 expression exhibited by a control sample. In certain specific embodiments, the keratin 17 expression value that corresponds with squamous cell carcinoma is exemplified by KRT17 staining in ≧8%, or between 5% and 10% of cells in a sample.

In yet another embodiment, the amount of KRT4 expression indicative of a subject having SCC includes, but is not limited to, a 5-10%, 10-20% decrease in expression compared to that of a control sample, or at least 20%, 30%, 40%, 50%, 60%, 70%, 80%, 90%, 100%, 200% or greater decrease in KRT4 expression when compared to that of a control sample, or at least a 0.25 fold, 0.5 fold, 1 fold, 1.5 fold, 2 fold, 3 fold, 4 fold, 5 fold, 10 fold, 11 fold or greater, decrease relative to the amount of KRT4 expression exhibited by a control sample. In certain embodiments, the keratin 4 expression level indicative of squamous cell carcinoma is exemplified by the presence of KRT4 staining in ≦6% or between 1% and 7% of the cells present in a sample.

### Prognostic Methods

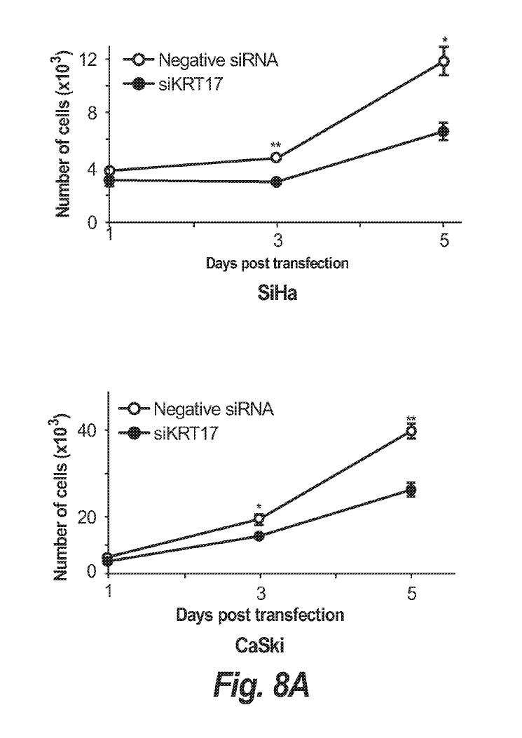

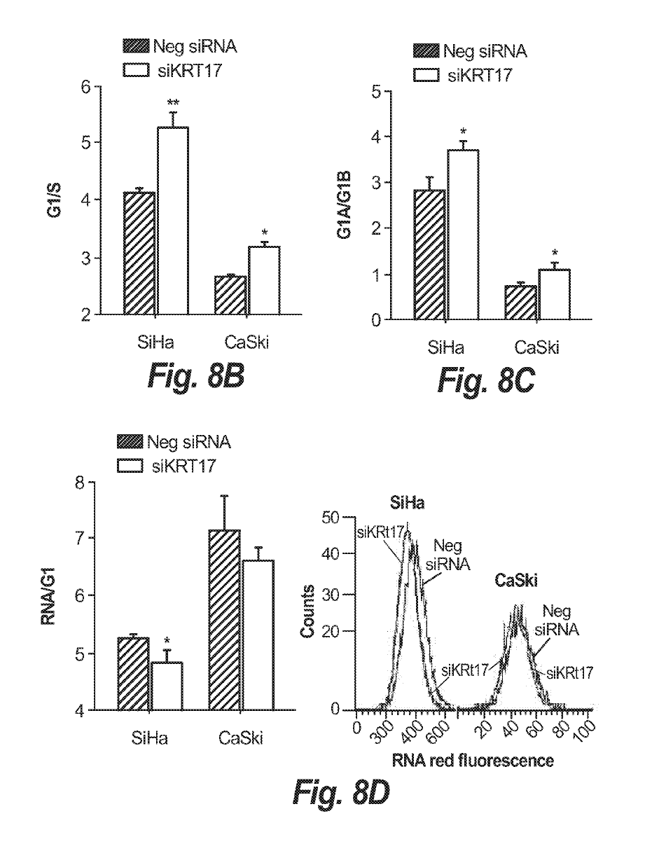

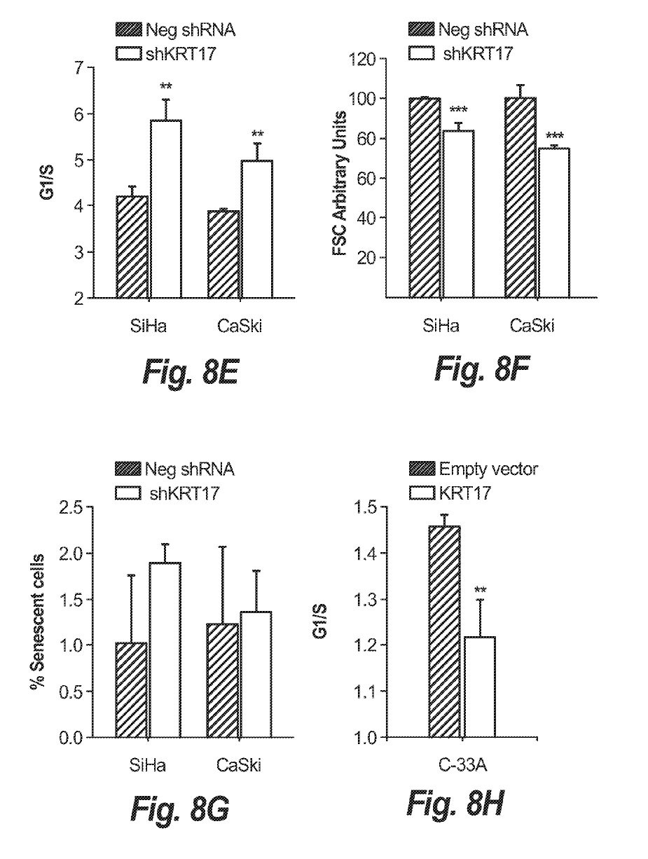

In view of keratin 17's utility as a biomarker for squamous cell carcinoma and/or SCC disease progression, the role of KRT17 was further characterized. The current disclosure shows that cell proliferation in several human cervical cancer cell lines (i.e., SiHa, CaSki, C-33A, HT-3, ME-180 and HeLa) and growth are well correlated to KRT17 expression. See, FIG. 8. More specifically, FIG. 8A of the present disclosure provides that the expression of KRT17 in human cervical cancer cell lines (e.g., SiHa, CaSki) leads to an increase in cellular proliferation, as evidenced in the significant increase in the number of cells found in cultures where KRT17 was expressed compared to cell samples where KRT17 expression was inhibited by RNA interference. Moreover, FIG. 8 B-E shows that the expression of KRT17 promotes cell cycle progression, while knockdown of KRT17 in human cervical cancer cell lines induces cell cycle arrest in G1-phase.

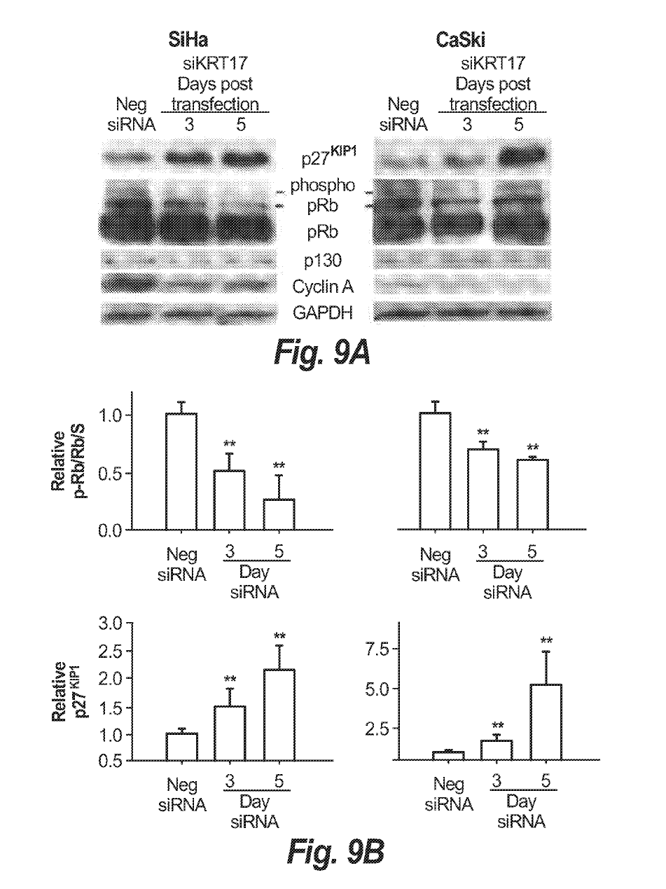

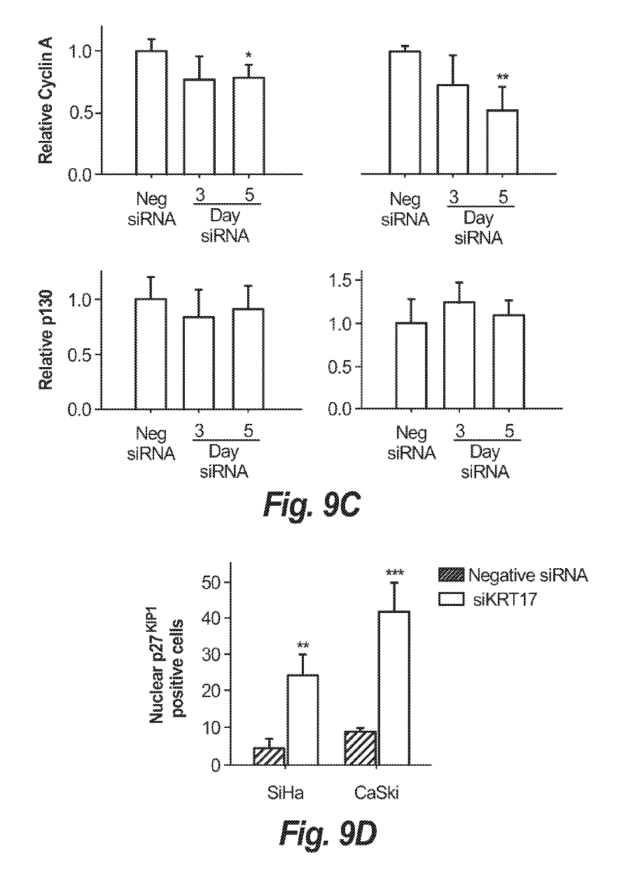

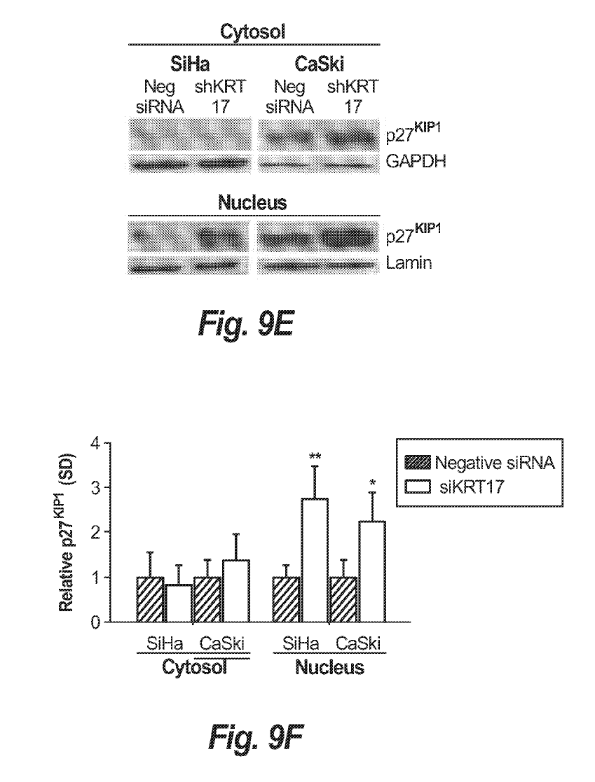

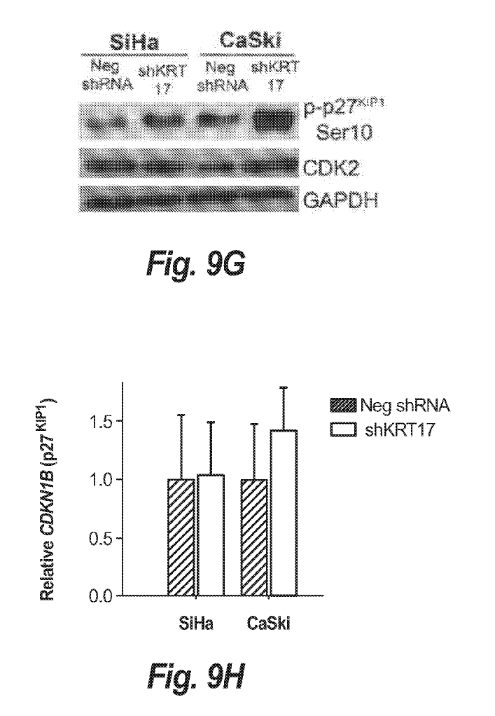

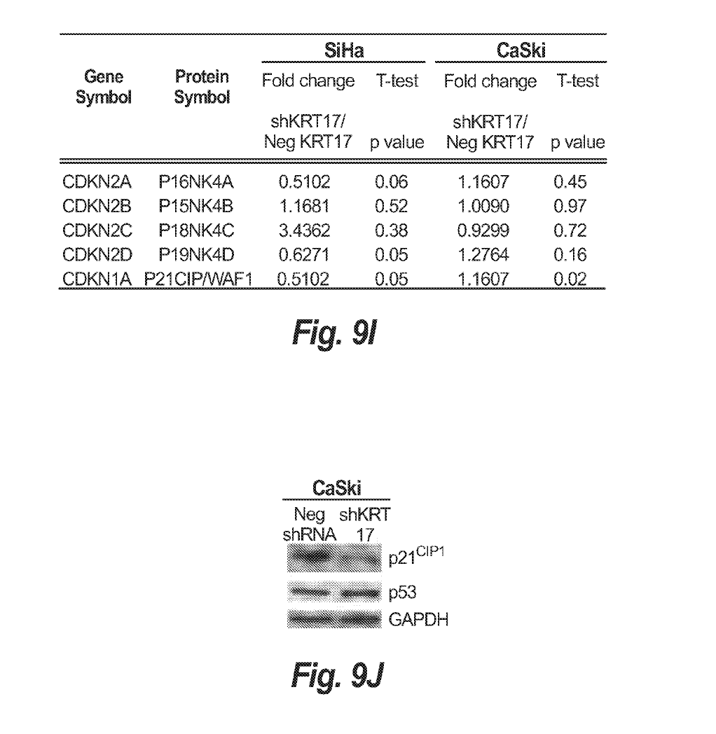

In view of the foregoing, cell growth was analyzed in cells expression KRT17 and compared to human cervical cancer cell lines whereby KRT17 expression was inhibited by short hairpin RNA against KRT17. See FIG. 8F. The cell growth data clearly show that cells expressing KRT17 are significantly larger than cells that do not express KRT17 or express normal levels of KRT17. The data provided herein further show that keratin 17 expression correlates to a reduction in nuclear p27Kip1, a protein that, when present in the nucleus, inhibits CDK2, which causes cell cycle arrest. See FIG. 9. Taken together, the current disclosures shows, for the first time, a novel role for KRT17 in cervical cancer progression, which lead the inventors of the instant disclosure to elucidate the role of KRT17 in determining treatment outcome and patient survival.

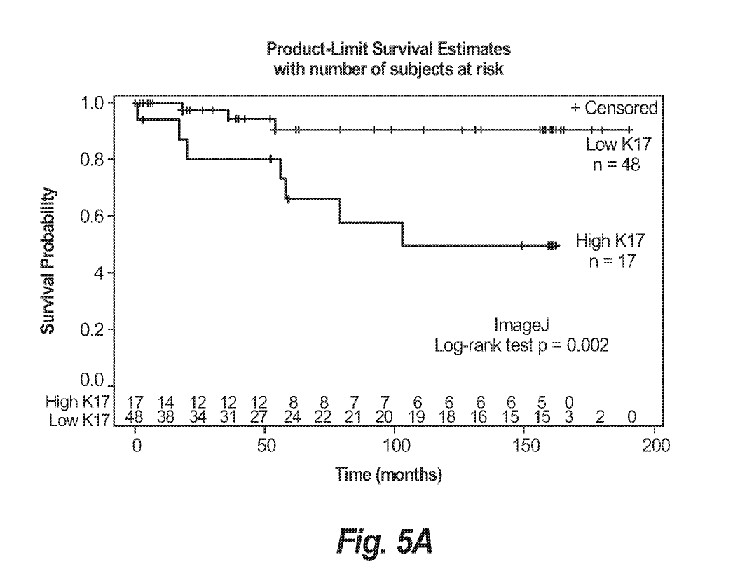

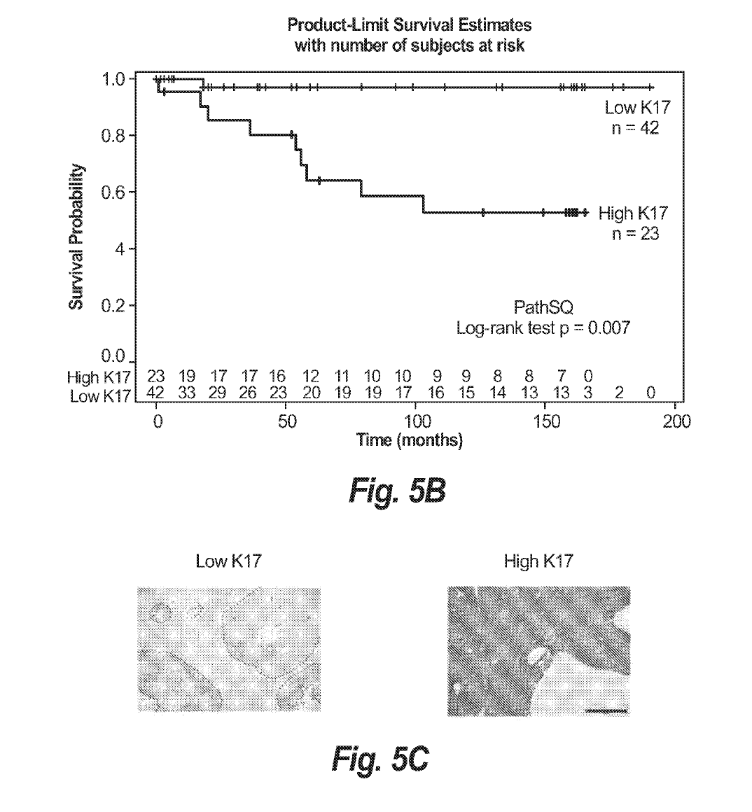

The instant disclosure further provides that the level of KRT17 expression is associated with poor survival of subjects having squamous cell carcinoma. More specifically, the data provided herein show that elevated expression of KRT17 in a subject diagnosed with squamous cell carcinoma indicates that the subject will have a reduced likelihood of survival and/or a negative treatment outcome when compared to a subject diagnosed with cervical cancer that does not exhibit an increase in KRT17 expression. See, for example, FIGS. 5-7.

In view of the foregoing, one aspect of the present disclosure provides methods for determining the likelihood of survival of a subject having cervical cancer, which includes obtaining a sample from a subject, detecting the level of KRT17 expression in the sample; and, optionally, further evaluating the KRT17 expression level in the sample obtained by comparing the level of KRT17 expression to the level of KRT17 expression in cancerous samples obtained from other subjects and/or a control sample.

In certain embodiments, a biological sample is obtained from the subject in question, i.e., a subject or patient diagnosed with HSIL or SCC. A biological sample, which can be used in accordance with the present methods, may be collected by a variety of means known to those of ordinary skill in the art. Non-limiting examples of sample collection techniques include; fine needle aspiration, surgical excision, endoscopic biopsy, excisional biopsy, incisional biopsy, fine needle biopsy, punch biopsy, shave biopsy and skin biopsy. Additionally, KRT17 expression can be detected from cancer or tumor tissue or from other body fluid samples such as whole blood (or the plasma or serum fractions thereof) or lymphatic tissue. In certain embodiments, the sample obtained from a subject is used directly without any preliminary treatments or processing, such as formalin-fixing, flash freezing, or paraffin embedding. In a specific embodiment, a biological sample can be obtained from a subject and processed by formalin treating and embedding the formalin-fixed sample in paraffin, and stored prior to evaluation by the instant methods.

In certain embodiments, after a suitable biological sample is obtained, the level of KRT17 expression in the sample can be determined using various techniques known by those of ordinary skill in the art. In specific embodiments of the current disclosure, KRT17 expression levels may be measured by a process selected from: immunohistochemistry (IHC), microscopy, q-RT-PCR, northern blotting, western blotting, enzyme-linked immunosorbent assays (ELISA), microarray analysis, or RT-PCR.

In a specific embodiment, immunohistochemical analysis of KRT17 is conducted on formalin-fixed, paraffin-embedded samples. Here, HSIL and/or squamous cell carcinoma samples from hematoxylin and eosin stained tissue sections can be dissected by laser capture microscopy. Formalin-fixed, paraffin-embedded tissue samples are then incubated in 50 mM Ammonium Bicarbonate with protease cocktails to facilitate the reverse of protein cross-linking. The samples can then be further processed by homogenization in urea. The protein concentration of KRT17 can then be determined by any suitable method known to one of skill in the art.

In a specific embodiment, KRT17 protein detection is carried out via tissue microarray. For example, tissue containing HSIL or squamous cell carcinoma can be obtained from paraffin blocks and placed into tissue microarray blocks. In certain embodiments, other sources of tissue samples can be used as control samples including, but not limited to, commercial tissue microarray samples, such as those obtained from HISTO-Array™, non-cancerous mucosal tissue or SCC tissue samples with known KRT17 expression levels. Tissue microarray slides for use in the current methods can then be processed, i.e., deparaffinized in xylene and rehydrated using an alcohol.

In certain embodiments, a sample can then be further processed by: incubation with a citrate buffer, applying hydrogen peroxide to block endogenous peroxidase, or by treating the sample with serum to block non-specific binding (e.g., bovine, donkey, human or horse serum). The samples can then be further incubated with primary antibodies against KRT17. Any antibody can be used against the KRT17 antigen including, but not limited to, mouse monoclonal-[E3] anti-human KRT17 antibody, polyclonal antibodies against human KRT17, a monoclonal antibody or polyclonal antibody against a mammalian KRT17 protein domain or epitope thereof. In certain embodiments, after incubation with the primary antibody, samples are processed by an indirect avidin-biotin-based immunoperoxidase method using biotinylated secondary antibodies, developed, and counter-stained with hematoxylin. Slides can then be analyzed for KRT17 expression using microscopy (e.g., fluorescent microscopy or light microscopy).

In certain specific embodiments, keratin expression is quantified by PathSQ method, a manual semi-quantitative scoring system, which quantifies the percentage of strongly stained cells, blinded to corresponding clinical data. In yet another embodiment, slides can be scored by the National Institutes of Health ImageJ 1.46, Java-based image processor software using the DAB-Hematoxylin (DAB-H) color deconvolution plugin. See Schneider C A, et al., Nat methods. (2012) 9:671-5.

In one embodiment KRT17 expression can be determined using enzyme-linked immunosorbent assays (ELISA). For example, a monoclonal antibody specific for KRT17 is added to the wells of microtiter strips or plates. Test samples obtained from a subject in question, a control SSC sample containing normal KRT17 protein expression levels, non-cancerous control samples, which exhibits no KRT17 expression, are provided to the wells. The samples are then incubated to allow the KRT17 protein antigen to bind the immobilized (capture) KRT17 antibody. The samples are then subjected to a washing with a buffer solution and subsequently treated with a detection antibody capable of binding by binding to the KRT17 protein captured during the first incubation. In certain embodiments, after removal of excess detection antibody, labeled antibody (e.g., anti-rabbit IgG-HRP) is added, which binds to the detection antibody to complete complex formation. After a third incubation and washing to remove all the excess labeled antibody, a substrate solution is added, which is acted upon by the bound enzyme to produce color. The intensity of this colored product is directly proportional to the concentration of total KRT17 protein present in the original sample. The amount of KRT17 protein present in a sample can then be determined by reading the absorbance of the sample and comparing to the control wells, and plotting the absorbance against control KRT17 expression levels using software known by those of ordinary skill in the art.

In yet another embodiment, KRT17 expression can be determined using reverse transcriptase PCR (RT-PCR) or quantitative-RT-PCR. More specifically, total RNA can be extracted from a sample by using a Trizol reagent. Reverse transcriptase PCR can then be performed using methods know by one of ordinary skill in the art. For example, RNA can be used as a template for cDNA synthesis and cDNA templates can then be mixed with gene-specific primers (i.e., forward, 5′-3′ primer sequence and reverse 3′-5′ sequence) for KRT17. Probe sequences for detection can also be added (e.g., Taqman or SYBR Green. Real-time quantitative PCR can then be carried out on each sample and the data obtained can be normalized to control levels of KRT17, as set forth in a control or normal sample. See, for example, Schmittgen, and Livak, Nature protocols (2008) 3: 1101-1108.

In a specific embodiment, samples mounted on slides and stained with KRT17 antibodies can be analyzed and scored by the National Institutes of Health ImageJ 1.46 (see Schneider C A, et al., Nat methods. (2012) 9:671-5) Java-based image processor software using the DAB-Hematoxylin (DAB-H) color deconvolution plugin (see Ruifrok A C, Johnston D A. Anal Quant Cytol Histol. (2001) 23:291-9) and/or by a manual semi-quantitative scoring system, which quantifies the percentage of strong-positively stained cells blinded to corresponding clinical data (PathSQ).

In preferred embodiments the level of KRT17 expression in a sample is determined by determining an ImageJ score and/or a PathSQ score for a subset of patients and choosing an appropriate level of KRT17 expression according to the lowest Akaike's information criteria in view of a Cox proportional-hazard regression model. In other embodiments, a low level of KRT17 expression is exemplified by the presence of KRT17 staining in less than 50% of the cells present in a sample. In yet another embodiment, a low level of KRT17 expression is indicated by the presence of KRT staining in less than 52% of the cells present in a sample or less than 52.5% of cells present in a sample. Conversely, a high level of KRT17 expression in a subject, which corresponds with a low incidence of survival beyond 5 years is indicated by the presence of KRT17 staining in at least 50% of the cells in a sample. In certain embodiments, a high level of KRT17 expression in a subject constitutes a sample with greater than 52% or greater than 52.5% of the cells in a sample staining positive for KRT17 protein.

Taken together, the current disclosure provides methods for determining the likelihood of survival of a subject that has been diagnosed with SCC and/or HSIL by analyzing the level of KRT17expression in a sample; and determining whether the level of KRT17 is highly overexpressed in the test sample. Whereby a highly level of KRT17 expression in squamous cell carcinoma identifies a subject as having the greatest risk for cervical cancer mortality.

### TERMINOLOGY

The term “peptide” or “protein” as used in the current disclosure refers to a linear series of amino acid residues linked to one another by peptide bonds between the alpha-amino and carboxy groups of adjacent amino acid residues. In one embodiment the protein is keratin 17 (KRT17). In yet another embodiment the protein is keratin 4 (KRT4).

The term “nucleic acid” as used herein refers to one or more nucleotide bases of any kind, including single- or double-stranded forms. In one aspect of the current disclosure a nucleic acid is DNA and in another aspect the nucleic acid is RNA. In practicing the methods of the current disclosure, nucleic acid analyzed (e.g., KRT4 or KRT17 RNA) by the present method is originated from one or more samples.

The term “keratin 17”, “K17” or “KRT17” as used herein refers to the human keratin, keratin, type II cytoskeletal 4 gene located on chromosome 17, as set forth in accession number NG_008625 or a product thereof, which encodes the type I intermediate filament chain keratin 17. Included within the intended meaning of KRT17 are mRNA transcripts of the keratin 17 cDNA sequence as set forth in accession number NM_000422, and proteins translated therefrom including for example, the keratin, type 1 cytoskeletal protein, 17 as set forth in accession number NP_000413 or homologs thereof.

The term “keratin 4”, “K4” or “KRT4” as used herein refers to the human keratin, type II cytoskeletal 4 gene located on chromosome 12, as set forth in accession number NG_007380.1 or a product thereof, which encodes the type II intermediate filament chain that is expressed in differentiated layers of the mucosal epithelia. Included within the intended meaning of KRT4 are mRNA transcripts of the keratin 4 cDNA sequence as set forth in accession number NM_0002272, and proteins translated therefrom including for example, the keratin, type II cytoskeletal protein, 4 as set forth in accession number NP_002263 or homologs thereof.

The phrase “subject”, “test subject” or “patient” as used herein refers to any mammal. In one embodiment the subject is a candidate for cancer diagnosis (e.g., squamous cell carcinoma) or an individual with cervical cancer or the presence of a pre-cancerous lesion, such as HSIL or LSIL. In certain embodiments, the subject has been diagnoses with SCC and the subject is a candidate for treatment thereof. The methods of the current disclosure can be practiced on any mammalian subject that has a risk of developing cancer or has been diagnosed with cancer. Particularly, the methods described herein are most useful when practiced on humans.

A “biological sample,” “test sample” or “sample(s)” as used in the instant disclosure can be obtained in any manner known to a skilled artisan. Samples can be derived from any part of a subject, including whole blood, tissue, lymph node or a combination thereof. In certain embodiments the sample is a tissue biopsy, fresh tissue or live tissue extracted from a subject. In other embodiments, the sample is processed prior to use in the disclosed methods. For example, a formalin-fixed, paraffin-embedded tissue sample isolated from a subject are useful in the methods of the current disclosure because formalin fixation and paraffin embedding is beneficial for the histologic preservation and diagnosis of clinical tissue specimens, and formalin-fixed paraffin-embedded tissues are more readily available in large amounts than fresh or frozen tissues.

A “control sample” “non-cancerous sample” or “normal sample” as used herein is a sample which does not exhibit elevated KRT17 and/or reduced KRT4 levels. In certain embodiments, a control sample does not contain cancerous cells (e.g., benign tissue components including, but not limited to, normal squamous mucosa, ectocervical squamous mucosa stromal cells, lymphocytes, and other benign mucosal tissue components). In another embodiment a control or normal sample is a sample from benign or cancerous tissues, that does not exhibit elevated KRT17 expression levels. Non-limiting examples of control samples for use in the current disclosure include, non-cancerous tissue extracts, surgical margins extracted from the subject, isolated cells known to have normal or reduced KRT17 levels, or samples obtained from other healthy individuals. In one aspect, the control sample of the present disclosure is benign tissue obtained from the subject in question.

The term “increase” or “greater” or “elevated” means at least more than the relative amount of an entity identified (such as KRT4 or KRT17 expression), measured or analyzed in a control sample. Non-limiting examples, include but are not limited to, a 5-10%, 10-20% increase over that of a control sample, or at least 20%, 30%, 40%, 50%, 60%, 70%, 80%, 90%, 100%, 200% or greater increase over that of a control sample, or at least a 0.25 fold, 0.5 fold, 1 fold, 1.5 fold, 2 fold, 3 fold, 4 fold, 5 fold, 10 fold, 11 fold or greater, increase relative to the entity being analyzing in the control sample.

The term “decrease” or “reduction” means at least lesser than the relative amount of an entity identified, measured or analyzed in a control sample. Non-limiting examples, include but are not limited to, 5-10%, 10-20% decrease compared to that of a control sample, or at least 20%, 30%, 40%, 50%, 60%, 70%, 80%, 90%, 100%, 200% or greater decrease when compared to that of a control sample, or at least a 0.25 fold, 0.5 fold, 1 fold, 1.5 fold, 2 fold, 3 fold, 4 fold, 5 fold, 10 fold, 11 fold or greater, decrease relative to the entity being analyzing in the control sample.

A “reduced level of KRT4 expression” as used in the current disclosure shall mean a decrease in the amount of KRT4 protein or peptide fragments thereof, or RNA present in a cell, organism or sample as compared to a control or normal level of KRT4 expression. In certain specific embodiments, the reduced level of keratin 4 expression indicative of squamous cell carcinoma is exemplified by the presence of KRT4 expression in ≦6% or between 1% and 7% of the cells present in a sample.

An “increased level of KRT17 expression” as used in the current disclosure shall mean an increase in the amount of KRT17 protein or peptide fragments thereof, or RNA present in a cell, organism or sample as compared to a control or normal level of KRT17 expression. In certain specific embodiments, the increased level of keratin 17 expression that corresponds with squamous cell carcinoma is exemplified by the presence of KRT17 expression in ≧8%, or between 5% and 10% of cells in a sample. In yet another embodiment, an increased level of KRT17 expression, which is indicative of lower patient survival, is indicated by the presence of KRT17 staining in at least 50% of the cells in a sample, or with greater than 52% or greater than 52.5% of the cells in a sample staining positive for KRT17.

### EXAMPLES

### Example 1

### Materials and Methods

Subject (Patient) Samples.

The study carried out included the analysis of 124 formalin-fixed paraffin-embedded surgical tissue blocks (Table 1). All surgical tissue blocks were obtained from subjects (patients) that underwent care from 1989 to 2011. The criteria for selection were (i) cases with pathology diagnosis of normal ectocervical squamous or unremarkable normal ectocervical squamous mucosa (normal ectocervical squamous mucosa), LSIL (CIN1), HSIL (CIN2/3), primary squamous cell carcinoma of the cervix (ii) age of subjects ≧18 years at time of diagnosis. Subjects diagnosed with cancer at other anatomic sites (i.e., outside of the cervix) were excluded from the study. In all cases, histologic review was performed by review of hematoxylin and eosin (H&E) stained slides to confirm that diagnostic tissue, as originally reported, was represented in the residual tissue block. Cases that were initially classified as CIN1 were reclassified as LSIL and cases that were reported as CIN2 or CIN3 were classified as HSIL. All other cases were classified as originally reported, without revision of the initial diagnoses. Cases that had insufficient residual tissue were excluded from the study. Squamous cell carcinomas were classified by: (i) clinical stage according to Edge S B and Compton C C. Annals of surgical oncology. (2010) 17:1471-4, (ii) tumor grade and (iii) lymph node status (Table 1). Survival data for each subject was obtained from the Stony Brook University Cancer Registry.

Cell Culture.

The human cervical cancer cell lines SiHa, CaSki, C-33A, HT-3, ME-180 and HeLa were obtained from the American Type Culture Collection (ATCC, Manassas, Va., USA) and cultured as recommended with RPMI1640, DMEM or McCoy's 5A medium (Gibco-Life Technologies) with 10% fetal bovine serum (Sigma-Aldrich, St Louis, Mo., USA). Cells were grown at 37° C. in a humidified atmosphere containing 5% CO₂. The medium was replaced every 48 hours.

Sample Preparation.

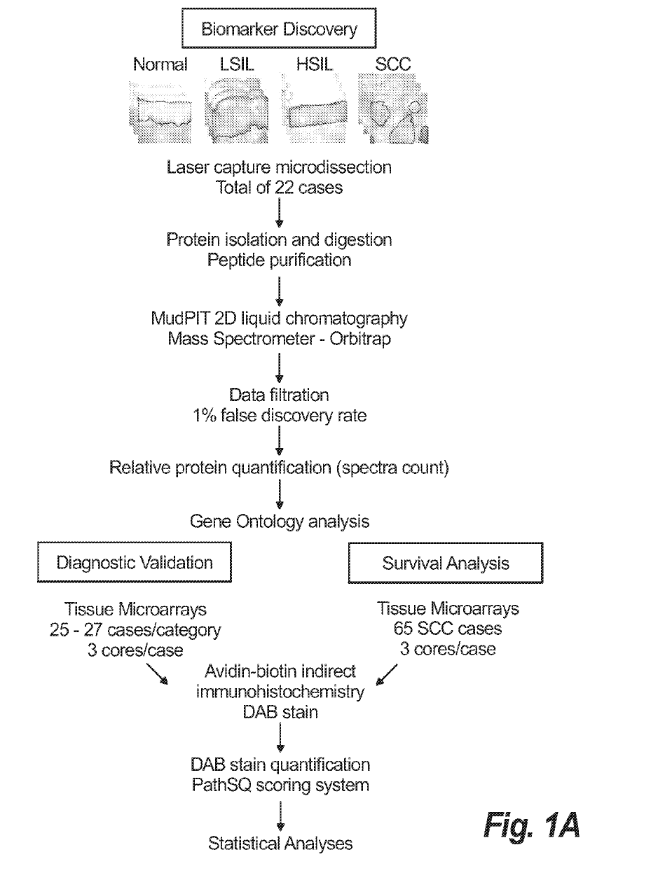

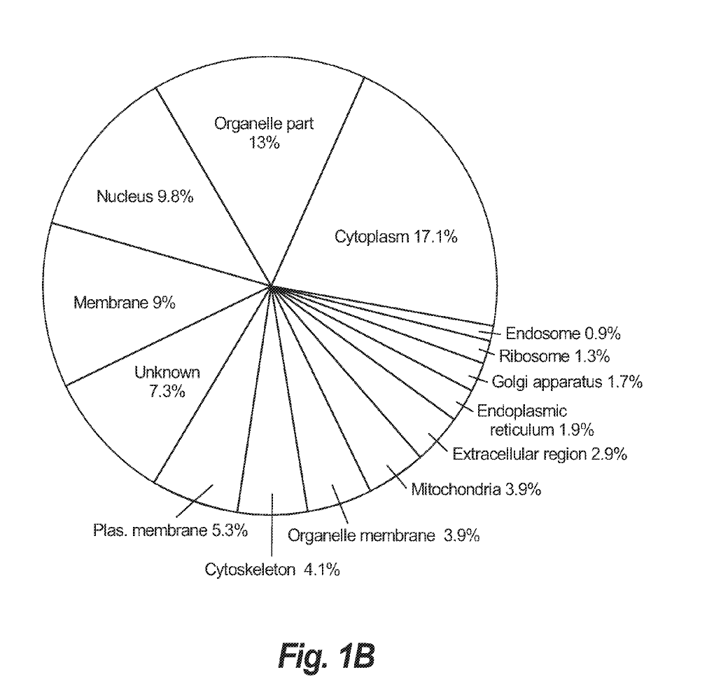

A total of 22 formalin-fixed paraffin-embedded tissue samples from all diagnostic categories were used for proteomic analysis. Or separately 74 formalin-fixed paraffin-embedded surgical tissue blocks provided from the UMass Memorial Medical Center. Normal cervical mucosa, LSIL, HSIL and squamous cell carcinoma from hematoxylin and eosin stained tissue sections were dissected by laser capture microscopy (Zeiss P.A.L.M.), collecting 540,000 to 650,000 cells from each diagnostic category. Dissected tissues were pooled from each diagnostic category for homogenization (FIG. 1). Formalin-fixed, paraffin-embedded tissues were first incubated in 50 mM Ammonium Bicarbonate (pH 9) with protease cocktails (Roche, Branford, Conn., USA) at 65° C. for 3 hours to facilitate the reverse of protein cross-linking. Then, tissues were homogenized in 4M urea in 50 mM ammonium bicarbonate (pH 7) with Invitrosol™ (Invitrogen, Carlsbad, Calif., USA) and RapiGest™ (Waters Corporation, Milford, Mass.) (17). The protein concentration was determined using an EZQ protein assay (Invitrogen, Carlsbad, Calif., USA).

Trypsin Digestion.

10 g of tissue lysates were diluted in 50 mM ammonium bicarbonate for trypsin digestion. Modified trypsin for sequencing grade (Promega, Fitchburg, Wis.) was added to each sample at a ratio of 1:30 enzyme/protein along with 2 mM CaCl₂ and incubated for 16 hours at 37° C. Following digestion, all reactions were acidified with 90% formic acid (2% final) to stop proteolysis. Then, samples were centrifuged for 30 minutes at 14,000 rpm to remove insoluble materials. The soluble peptide mixtures were collected for liquid chromatography-tandem mass analysis.

Multidimensional Chromatography and Tandem Mass Spectrometry.

Peptide mixtures were pressure-loaded onto a 250 μm inner diameter (i.d.) fused-silica capillary packed first with 3 cm of 5 μm strong cation exchange material (Partisphere SCX, Whatman), followed by 3 cm of 10 μm C18 reverse phase (RP) particles (Aqua, Phenomenex, Calif., USA). Loaded and washed microcapillaries were connected via a 2 μm filtered union (UpChurch Scientific) to a 100 μm i.d. column, which had been pulled to a 5 μm i.d. tip using a P-2000 CO₂ laser puller (Sutter Instrument, Novato, Calif., USA), then packed with 13 cm of 3 μm C18 RP particles (Aqua, Phenomenex, Calif., USA) and equilibrated in 5% acetonitrile, 0.1% formic acid (Buffer A). This split-column was then installed in line with a Nano-liquid chromatography Eskigent high-performance liquid chromatography pump. The flow rate of channel 2 was set at 300 nl/min for the organic gradient. The flow rate of channel 1 was set to 0.5 μl/min for the salt pulse. Fully automated 13-step chromatography runs were carried out. Three different elution buffers were used: 5% acetonitrile, 0.1% formic acid (Buffer A); 98% acetonitrile, 0.1% formic acid (Buffer B); and 0.5 M ammonium acetate, 5% acetonitrile, 0.1% formic acid (Buffer C). In such sequences of chromatographic events, peptides are sequentially eluted from the SCX resin to the RP resin by increasing salt steps (increase in Buffer C concentration), followed by organic gradients (increase in Buffer B concentration). The last chromatography step consisted of a high salt wash with 100% Buffer C followed by acetonitrile gradient. The application of a 2.5 kV distal voltage electrosprayed the eluting peptides directly into an LTQ-Orbitrap XL mass spectrometer equipped with a nano-liquid chromatography electrospray ionization source (Thermo Finnigan, San Jose, Calif., USA). Full mass spectrometry spectra were recorded on the peptides over a 400 to 2000 m/z range by the Orbitrap followed by five tandem mass events sequentially generated by LTQ in a data-dependent manner on the first, second, third, and fourth most intense ions selected from the full mass spectrometry spectrum (at 35% collision energy). Mass spectrometer scan functions and high-performance liquid chromatography solvent gradients were controlled by the Xcalibur data system (Thermo Finnigan, San Jose, Calif., USA).

Database Search and Interpretation of Tandem Mass Spectrometry Datasets.

Spectra from triplicate runs were merged from each category for data analysis. Tandem mass spectra were extracted from raw files, and a binary classifier, previously trained on a manually validated data set, was used to remove the low-quality tandem mass spectra. The remaining spectra were searched against a human protein database containing 69,711 protein sequences downloaded as FASTA-formatted sequences from UniProtKB (see UniProtConsortium. Reorganizing the protein space at the Universal Protein Resource (UniProt). Nucleic Acids Res. 2012; 40: D71-5) and 124 common contaminant proteins, for a total of 69,835 sequence entries. To calculate confidence levels and false positive rates, a decoy database was used containing the reverse sequences of 69,835 proteins appended to the target database (see Elias J E and Gygi S P. Nat. Methods. 2007; 4: 207-14), and the SEQUEST algorithm (see Eng J K, et al., Analytical Chemistry. 1995; 67: 1426-36; and Ashburner M, et al. Nature Genet. 2000; 25: 25-9) to find the best matching sequences from the combined database. S EQUEST searches were done using the Integrated Proteomics Pipeline (IP2, Integrated Proteomics Applications, San Diego, Calif., USA) on Intel Xeon X5450 X/3.0 PROC processor clusters running under the Linux operating system. The peptide mass search tolerance was set to 50 ppm. No differential modifications were considered. No enzymatic cleavage conditions were imposed on the database search, therefore the search space included all candidate peptides whose theoretical mass fell within the 50 ppm mass tolerance window, despite their tryptic status.

The validity of peptide/spectrum matches was assessed in Scaffold software (see Lundgren D H, et al., Curr Protoc Bioinformatics. (2009) Chapter 13:Unit 13 3) using SEQUEST-defined parameters, the cross-correlation score (XCorr) and normalized difference in cross-correlation scores (DeltaCN). The search results were grouped by charge state (+1, +2, and +3) and tryptic status (fully-, half-, and non-tryptic), resulting in 9 distinct sub-groups. In each one of the sub-groups, the distribution of XCorr and DeltaCN values for (a) direct and (b) decoy database hits was obtained, and the two subsets were separated by quadratic discriminant analysis. Outlier points in the two distributions (for example, matches with very low Xcorr but very high DeltaCN) were discarded. Full separation of the direct and decoy subsets is not generally possible; therefore, the discriminant score was set such that a false positive rate of 1% was determined based on the number of accepted decoy database peptides. This procedure was independently performed on each data subset, resulting in a false positive rate independent of tryptic status or charge state. In addition, a minimum sequence length of seven amino acid residues was required, and each protein on the final list was supported by at least two independent peptide identifications unless specified. These additional requirements, especially the latter, resulted in the elimination of most decoy database and false positive hits, as these tended to be overwhelmingly present as proteins identified by single peptide matches. After this last filtering step, the false identification rate was reduced to below 1%. Global normalization was performed by Scaffold software (Proteome Software, Inc. Portland, Oreg.). Gene Ontology (see Ashburner M, et al., Nature Genet. (2000) 25:25-9) was used to determine the subcellular localization of identified proteins.

Diagnostic Validation by Immunohistochemical Analysis.

To validate the proteomic profile data, tissue microarrays of 25-27 cases per diagnostic category were constructed (FIG. 1). Each case contained up to three core replicates, with the exception of 12 LSIL cases, which contained only one core due to the small size of the lesions. Slides were reviewed and areas containing normal cervical mucosa, LSIL, HSIL and squamous cell carcinoma were marked on glass slides. Three mm punches of tissue were used as samples that were then taken from the corresponding regions of the paraffin blocks and placed into tissue microarray blocks. In addition, a commercial tissue microarray containing 40 additional squamous cell carcinoma cases from HISTO-Array™ tissue arrays (IMGENEX, San Diego, Calif., USA) was purchased. After incubation at 60° C. for 1 h, tissue microarray slides were deparaffinized in xylene and rehydrated using graded alcohols. Antigen retrieval was performed in citrate buffer (20 mmol, pH 6.0) at 120° C. for 10 minutes in a decloaking chamber. Endogenous peroxidase was blocked by applying 3% hydrogen peroxide for 5 minutes. Sections were subsequently blocked in 5% horse serum. Primary antibodies used were: mouse monoclonal-[E3] anti-human KRT17 antibody (ab75123, Abcam, Cambridge, Mass., USA; 4° C. overnight) and mouse monoclonal-[6B10] anti-human KRT4 antibody (vp-c399, Vector Laboratories, Burlingame, Calif.; 1:150 1 h room temperature). After incubation with the primary antibody, slides were processed by an indirect avidin-biotin-based immunoperoxidase method using biotinylated horse secondary antibodies (R.T.U. Vectastain Universal Elite ABC kit; Vector Laboratories, Burlingame, Calif., USA), developed in 3,3′ diaminobenzidine (DAB) (K3468, Dako, Carpentaria, Calif., USA), and counter-stained with hematoxylin. Negative controls were performed on all cases using an equivalent concentration of a subclass-matched mouse immunoglobulin, generated against unrelated antigens, in place of primary antibody. Slides were scored by PathSQ, a manual semi-quantitative scoring system, which quantifies the percentage of strongly stained cells, blinded to corresponding clinical data.

Scoring of Keratin Protein Expression.

Slides were scored by the National Institutes of Health ImageJ 1.46 (see Schneider C A, et al., Nat methods. (2012) 9:671-5, the contents of which is incorporated herein by reference) Java-based image processor software using the DAB-Hematoxylin (DAB-H) color deconvolution plugin (see Ruifrok A C, Johnston D A. Anal Quant Cytol Histol. (2001) 23:291-9, the contents of which is incorporated herein by reference) and by a manual semi-quantitative scoring system, which quantifies the percentage of strong-positively stained cells blinded to corresponding clinical data (PathSQ).

RT-PCR and qRT-PCR.

Total RNA was extracted with Trizol reagent (Invitrogen) following the manufacturer's protocol. Reverse transcriptase PCR was performed with Reverse Transcription System (Promega, Madison, Wis.). In all, 1 μg of RNA was used as a template for cDNA synthesis. cDNA templates were mixed with gene-specific primers for KRT17, CDKN2A (p16^{INK4a}), CDKN2B (p15^{INK4b}), CDKN2C (p18^{INK4c}), CDKN2D (p19^{INK4d}), CDKNIA (p21^{CIP1/WAF1}), CDKN1B (p27^{KIP1}), COPS5 (JAB1), GAPDH, β-actin and 18S. Taqman 2× universal PCR master mix or SYBR Green PCR Master Mix (Applied Biosystems) were used depending on the detection system. Applied Biosystems 7500 Real-Time PCR machine was used for qRT-PCR and programmed as: 95° C., 10 min; 95° C., 15 s; 60° C., 1 min and repeated for 40 cycles. Data was normalized by the level of expression in each individual sample as described in Schmittgen and Livak, Nature protocols 2008 3, 1101-1108, the contents of which is incorporated herein by reference.

Classification of High/Low K17 Expression in Cervical Cancer by ImageJ and PathSQ Scoring.

To display Kaplan-Meier curves of overall survival, the SCC cases were further divided into two groups according to KRT17's (K17) expression level, high K17 level vs. low K17 level, measured by ImageJ and PathSQ. The best cut-off points for both scoring methods were chosen according to the lowest Akaike's information criterion (AIC) from a Cox proportional-hazard regression model. A data-driven cutoff point of 163 (74^{th} percentile of total cases) in ImageJ score and 52.5% of PathSQ score (64^{th} percentile of total cases) were used to classify patients into two groups. High level of K17 (high K17), ImageJ score ≧163 or PathSQ score ≧52.5% and low level of K17 (low K17)<163 or <53% ImageJ and PathSQ score, respectively. In fact, any cut-off point within the interval of 161-165 (72^{nd}-75^{th} percentile, respectively) of ImageJ score or in the interval of 52-53 (63^{rd} and 65^{th} percentile, respectively) resulted in the same AIC values for Cox proportional hazard models. The midpoints of the Cox proportional hazard models 163 and 52.5% (reported as >50%) were used in the Kaplan-Meier curves of overall survival in SCC patients. Log-rank test was used to compare overall survival between SCC patients with high K17 levels and low K17 levels. The association between overall survival and other SCC factors (age, stage, grade and lymph node status) were studied through Kaplan-Meier estimate and log-rank tests. Hazard ratio (HR) and 95% CI were calculated based on Cox proportional hazard regression models. Statistical significance was set at 0.05 and analysis was done using SAS 9.3 (SAS Institute, Inc., Cary, N.C.) and SigmaPlot 11 (Systat Software, San Jose, Calif.).

In certain embodiments, the unit of measurement for immunohistochemical analysis was each core and the average PathSQ score of all cores was used for statistical analyses. The score differences between diagnostic categories were determined by Kruskal-Wallis or Wilcoxon rank-sum test. Receiver operating curves and the area under the curve were calculated to evaluate biomarker potential to discriminate different diagnostic categories based on logistic regression models. The optimal cut-off value from receiver operating curves was determined using Youden's index. See Youden W J. Cancer. (1950) 3:32-5, the contents of which is incorporated herein by reference. For keratin 4 (KRT4), the optimal cut-off value in the resultant receiver operating curve corresponded to ≧6% of positive cells, while for keratin 17 (KRT17), the optimal cut-off value in the resultant receiver operating curve corresponded to ≧8% of positive cells for PathSQ score. Sensitivity, specificity, positive predictive value, negative predictive value, and misclassification rates were calculated corresponding to the optimal cutoff values. Pearson's correlation coefficient was used to evaluate the correlation between KRT17 expression and other quantitative variables such as age of patient and time of tissue storage. Overall survival was defined from the time of surgery to death or last follow-up if still alive. The association between KRT17 expression and overall survival was estimated through univariate Cox proportional hazard models. Assumption for Cox proportional hazard model was confirmed.

Small-Interference RNA and Short-Hairpin RNA.

For transient transfection, ON-TARGETplus Human KRT17 (3872) small-interference RNAs (siRNA)-SMART pool (Thermo Scientific, Waltham, Mass., USA) of 4 siRNAs were used to knockdown KRT17 expression (siKRT17). The following KRT17 siRNA sequences were used to knockdown KRT17 expression: (5′-3′) AGAAAGAACCGGUGACCAC (SEQ ID NO: 1), CGUCAGGUGCGUACCAUUG (SEQ ID NO: 2), GGUCCAGGAUGGCAAGGUC (SEQ ID NO: 3), GGAGAGGAUGCCCACCUGA (SEQ ID NO: 4). ON-TARGETplus Non-targeting Control siRNAs (Thermo Scientific, Waltham, Mass., USA) were used as RNA interference control (Negative siRNA). siRNAs were transfected into cancer cells using Oligofectamine™ 2000 (Life Technologies, Grand Island, N.Y., USA) according to the standard protocol. For stable knockdown of KRT17, three GIPZ Lentiviral shRNA (GE Dharmacon Lafayette, Colo., USA) were used to screen for best knockdown efficiency. The following KRT shRNA sequences were used to knockdown KRT17 expression: (5′-3′) sh1-TCTTGTACTGAGTCAGGTG (SEQ ID NO: 5), sh2-TCTTTCTTGTACTGAGTCA (SEQ ID NO: 6), and sh3-CTGTCTCAAACTTGGTGCG (SEQ ID NO: 7). Negative GIPZ lentiviral shRNA controls were used as negative shRNA. Lentivirus production was carried out following manufactures' protocol. After cancer cell transduction, cells were selected with 10 g/ml, and stable clones were produced for each cell line.

Cell Proliferation, Cell Cycle Analysis and Senescence Assay.

Twenty-four hours after transient transfection, SiHa and CaSki cells were seeded in 96-well plates at 4000 cells/well. The cell proliferation assay was performed on days 1, 3 and 5 by incubating 10 μl WST-1 (Roche Applied Science, Mannheim, Germany) in the culture medium for 2 h and reading the absorbance at 450 and 630 nm. The cell proliferation rate was calculated by subtracting the absorbance at 450 nm from the absorbance at 630 nm. A cell number absorbance curve was performed to calculate cell per well. Cell cycle analysis was performed by flow cytometry using propidium iodine and acridine orange stains. Three days or two weeks after transient and stable transfections, respectively, cells were harvested and resuspended at 0.5-1×10⁶ cells/ml in modified Krishan buffer with 0.02 mg/ml RNase H (Invitrogen) and 0.05 mg/ml propidium iodide (Sigma-Aldrich). Results were calculated with Modfit LT software version 3 (Verity Software House, Topsham, Me., USA). For acridine orange cell cycle stain and analyses were performed as previously described (Darzynkiewicz et al., 1980; El-Naggar, 2004). All samples were analyzed in FACSCalibur™ (Becton Dickinson) at the Research Flow Cytometry core at Stony Brook University. The Senescence β-galactosidase staining kit (Cell Signaling, Danvers, Mass., USA #9860) was used to determine percentage of senescent cells following the manufactures' instructions.

Serum Starvation Release, Cycloheximide Chase and Leptomycin B Treatment.

For protein stability analysis, cells were plated into 60-mm dishes at 50% confluence and serum starved for 48 h. After serum starvation, cell were restimulated with DMEM containing 20% FBS and cycloheximide at 40 μg/ml (CHX, catalog no. 239764; Calbiochem). At the indicated time points, whole cell extracts were prepared and western blotted.

Western Blotting and Extraction of Nuclear Proteins.

Whole cell protein samples were collected with RIPA buffer (Sigma-Aldrich) and subsequently sonicated. Nuclear and cytoplasmic proteins were extracted by NE-PER™ Protein Extraction Reagent (Pierce) according to the manufacturer's instructions. Protein concentration was determined by the BCA protein assay (Pierce). Equal amounts of samples were loaded to sodium dodecyl sulfate polyacrylamide gel electrophoresis and transferred to polyvinylidene difluoride membrane. The membranes were blocked with 5% non-fat milk in TBS/0.5% Tween-20 (TBS-T) at room temperature for 30 min, then probed with: mouse anti-keratin 17 antibody (Cat #sc-101461, Santa Cruz Biotechnology, Santa Cruz, Calif.), mouse anti-human p27^{KIP1} antibody (Cat #610242, BD transduction Labs), rabbit anti-human pRB antibody (Cat #9313S, Cell Signaling, Danvers, Mass., USA), rabbit anti-cyclin Dl (Cat #2978S, Cell Signaling, Danvers, Mass., USA), rabbit anti-SKP2 (Cat #2652P, Cell Signaling, Danvers, Mass., USA), rabbit anti-phospho p27^{KIP1} Ser10 (Cat #sc-12939-R, Santa Cruz Biotechnology, Santa Cruz, Calif.), mouse anti-JAB1 (Cat #sc-13157, Santa Cruz Biotechnology, Santa Cruz, Calif.), mouse anti-HPV16 E6/18E6 (Cat #sc-460, Santa Cruz Biotechnology, Santa Cruz, Calif.), mouse anti-HPV16 E7 (Cat #sc-6981, Santa Cruz Biotechnology, Santa Cruz, Calif.), rabbit anti-cyclin A (Cat #sc-751 Santa Cruz Biotechnology, Santa Cruz, Calif.), mouse anti-RNF123 (KPC1) (Cat #sc-101122 Santa Cruz Biotechnology, Santa Cruz, Calif.), rabbit anti-UBE3A (Cat #AP2154B ABGENT, San Diego, Calif., USA), rabbit anti-p130 (Cat #sc-317, Santa Cruz Biotechnology, Santa Cruz, Calif.), rabbit anti-phospho keratin 17 Ser44 (Cat #3519S, Cell Signaling, Danvers, Mass., USA), rabbit anti-cytokeratin 17 (Cat #ab 109725 Abcam, Cambridge, Mass., USA), mouse anti-p53 antibody (Cat #sc-126, Santa Cruz Biotechnology, Santa Cruz, Calif., USA), mouse anti-human p21 antibody (Cat #2946, Cell Signaling, Danvers, Mass., USA), mouse anti-GAPDH antibody (Cat #sc-365062, Santa Cruz Biotechnology, Santa Cruz, Calif., USA), mouse anti-human α-tubulin antibody (Cat #05-829, Millipore, Temecula, Calif., USA), mouse anti-Lamin B1 (Cat #ab90576 Abcam, Cambridge, Mass., USA) overnight at 4° C. Goat anti-rabbit and anti-mouse and rabbit anti-goat horseradish peroxidase-conjugated secondary antibodies (Jackson Immunoresearch, West Grove, Pa., USA) were used at 1:5000. Horseradish peroxidase activity was detected with SuperSignal West Pico Chemiluminescent Substrate (Thermo Scientific, Waltham, Mass., USA) and visualized in an UVP Bioimaging system (Upland, Calif., USA). Expression levels were quantified using ImageJ software (National Institute of Health, Bethesda, Mass., USA), and normalized to loading controls as shown in FIG. 9.

### Example 2

### Biomarker Discovery and Candidate Selection

Lesional epithelial cells from 22 formalin-fixed paraffin-embedded tissues, including normal cervical mucosa, LSIL, HSIL and squamous cell carcinoma were processed by laser capture microdissection for proteomic analysis. Collected cells from multiple patients in each category were pooled to identify the most robust and consistent differences in protein abundance. Proteins were extracted from formalin-fixed paraffin-embedded tissues using mass spectrometry-compatible lysis buffer and analyzed using a high-resolution mass spectrometer, LTQ-OrbitrapXL. Using the 2D liquid chromatography-tandem mass analysis methods known to one of ordinary skill in the art, we identified 1750 proteins at 1% false discovery rate and derived relative quantification of these proteins among the categories using the spectral counting method (data not shown). See Liu H, et al., Anal Chem. (2004) 76: 4193-201. To examine the comprehensive sampling of formalin-fixed paraffin-embedded tissues by shotgun proteomic analysis, we assessed the cellular localization of identified proteins by the Gene Ontology database and showed that proteins were identified from a diverse range of subcellular locations supporting the utility of analyzing formalin-fixed paraffin-embedded tissues (FIG. 1b). To select candidate biomarkers, we first selected proteins with at least two-fold differences based on spectral counts among diagnostic categories and narrowed down this list further by selecting protein expression profiles indicative of disease progression. Based on these criteria, two candidate biomarkers KRT17 and KRT4 were selected for further validation. These two proteins show an opposite trend in the progression of normal to squamous cell carcinoma. KRT17 shows an increased expression from normal to LSIL, HSIL and to squamous cell carcinoma whereas KRT4 shows a decreased expression in the progression of normal to squamous cell carcinoma (data not shown).

### Example 3

### Keratin 4 and Keratin 17 as Diagnostic Markers

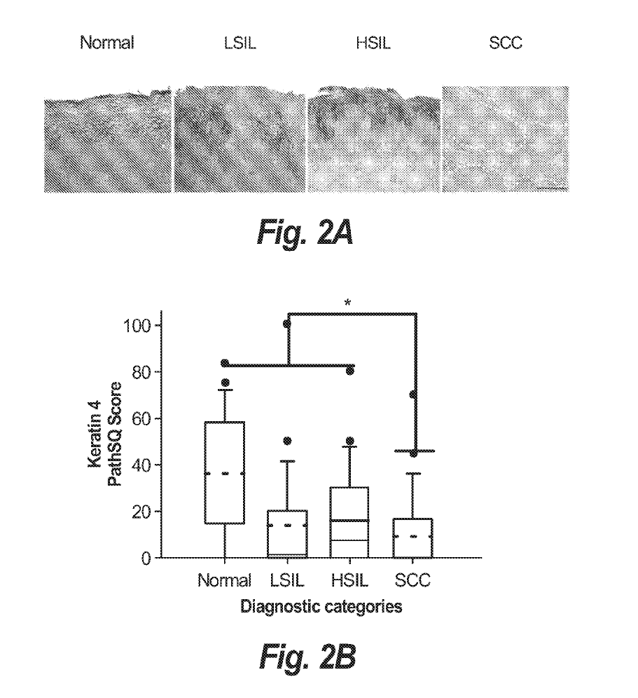

To determine the diagnostic values of KRT4 and KRT17 in one or more diagnostic categories, immunohistochemical staining was performed for KRT4 and KRT17 on tissue microarrays of archived patient tissues from four diagnostic categories: normal, LSIL, HSIL, squamous cell carcinoma. Immunostained slides were scored by PathSQ, which quantifies the percentage of strong-positively stained cells. Immunohistochemical analysis for KRT4 showed cytoplasmic expression in normal, LSIL and in some HSILs but was significantly reduced in squamous cell carcinomas (FIG. 2A-B). The loss of KRT4 had a sensitivity of 68% (95% CI: 46-85%) and specificity of 61% (95% CI: 49-72%) to distinguish squamous cell carcinoma from other diagnostic categories (Table 2). The positive predictive value, negative predictive value and area under the curve for the receiver operating curve model and misclassification rate are included in Table 2. According to the PathSQ cut-off value (≧6% of positive cells), 84% of normal cases, 44% of LSILs, 55% of HSILs and 32% of squamous cell carcinoma cases were positive for KRT4.

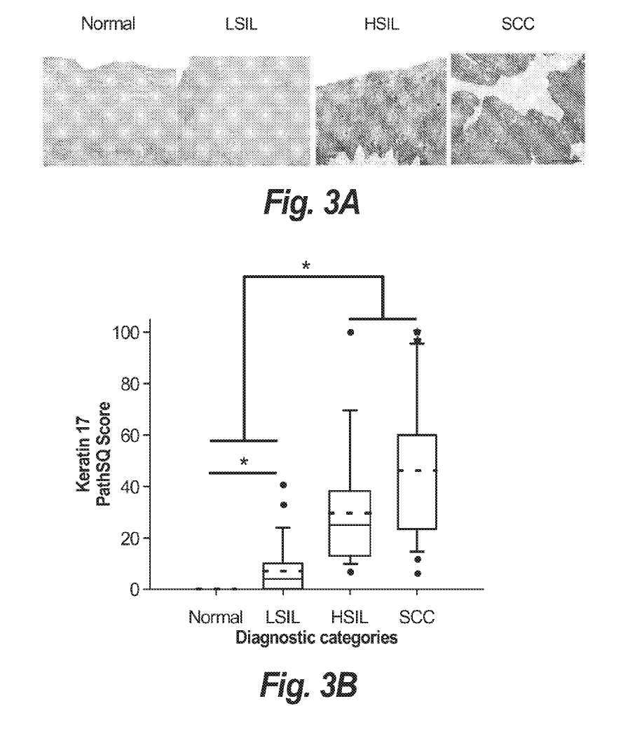

KRT17 immunohistochemical staining demonstrated a reciprocal pattern of cytoplasmic expression compared to that seen in KRT4; KRT17 was detected in most HSILs and squamous cell carcinomas but was generally detected at negligible levels in normal squamous mucosa, including ectocervical squamous mucosa, and LSIL (FIG. 3a-b). KRT17 had a sensitivity of 94% (95% CI: 73-94%) and specificity of 86% (95% CI: 73-94%) to distinguish HSIL/squamous cell carcinoma from normal mucosa/LSIL) (Table 2). The positive predictive value, negative predictive value, area under the curve and misclassification error rate values are included in Table 2. Based on the PathSQ cut-off value (≧8% of positive cells), all normal cases are negative, 27% of LSIL cases were positive and 96% of HSIL cases and 92% of squamous cell carcinoma cases were positive. Thus, our results suggest that KRT17 expression can distinguish patients with malignant lesions (HSIL or squamous cell carcinoma) with both high sensitivity and specificity from patients with non-malignant transient infections (LSIL) or healthy individuals with normal cervical mucosa.

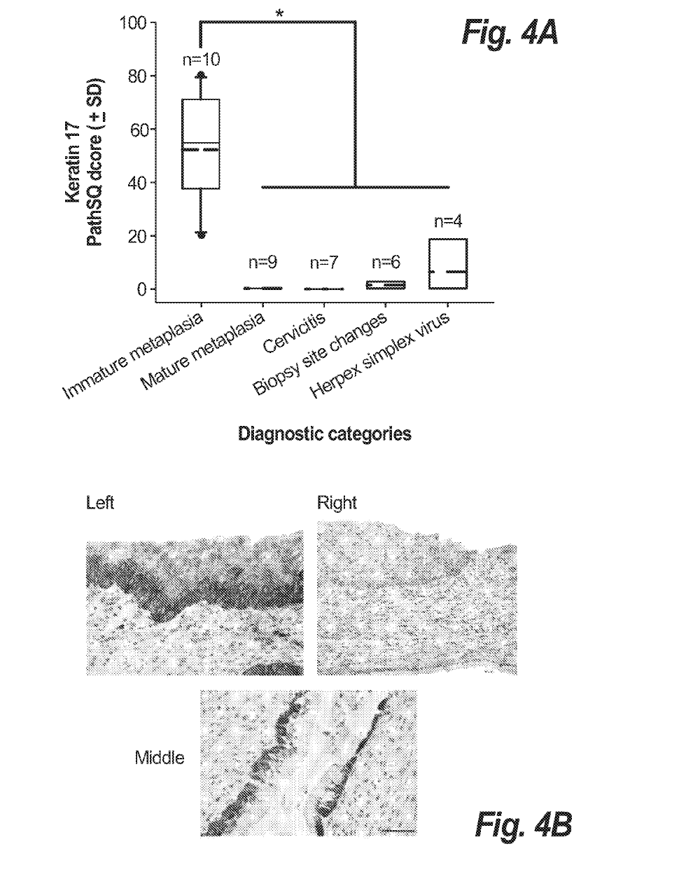

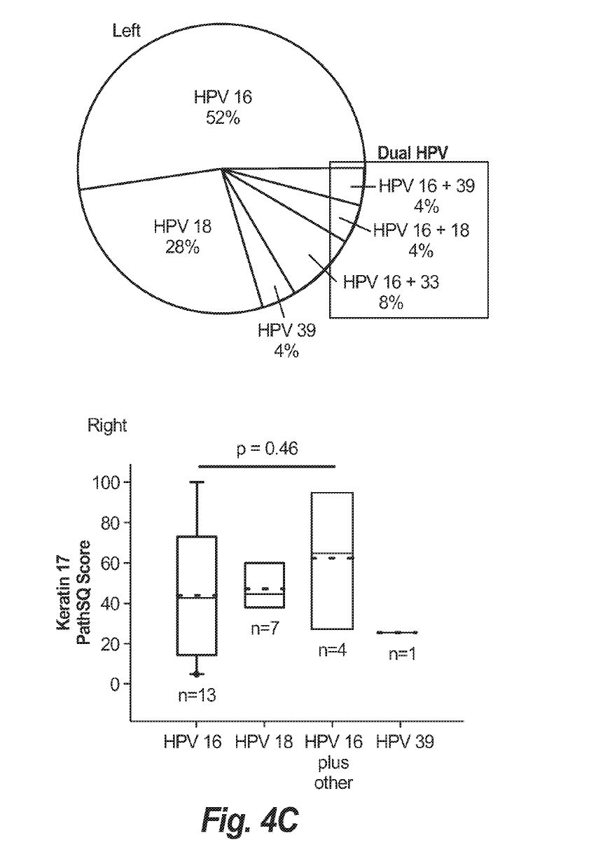

Next, disease-independent parameters were examined, including patient age and storage time of tissues to determine if any factor influenced the reliability of KRT17 as a biomarker for HSIL and squamous cell carcinoma cases. No significant correlation between KRT17 expression and the age of patients or length of tissue storage was found (r=0.02 and r=−0.40, with p-values >0.05, respectively). Furthermore, no statistically significant change of KRT17 expression was found in cases with cervicitis, mature squamous metaplasia, biopsy site changes (wound healing), or herpes simplex virus infection (FIG. 4A). KRT17, however, was detected in immature squamous metaplasia (FIG. 4A-B) and in endocervical reserve cells. From 17 cases with endocervical mucosa, 70% (12/17) had positive staining in reserve cells. Lastly, there was no statistically significant correlation between the KRT17 expression and different high-risk HPV types in squamous cell carcinoma patients (FIG. 4C).

### Example 4

### Keratin 17 as a Prognostic Biomarker for Patient Survival

Given the high sensitivity and specificity of KRT17 to distinguish high-grade lesions from normal mucosa and LSIL, additional squamous cell carcinoma cases were further examined to determine if KRT17 had a prognostic value for patient survival. Based on Cox proportional hazard model, KRT17 expression was significantly associated with reduced overall survival in squamous cell carcinoma patients (p=0.009). The midpoint of the Cox proportional hazard models strong staining in ≧50% of tumor cells was used as the threshold to separate squamous cell carcinoma cases for overall patient survival in the Kaplan-Meier curves (FIG. 5).

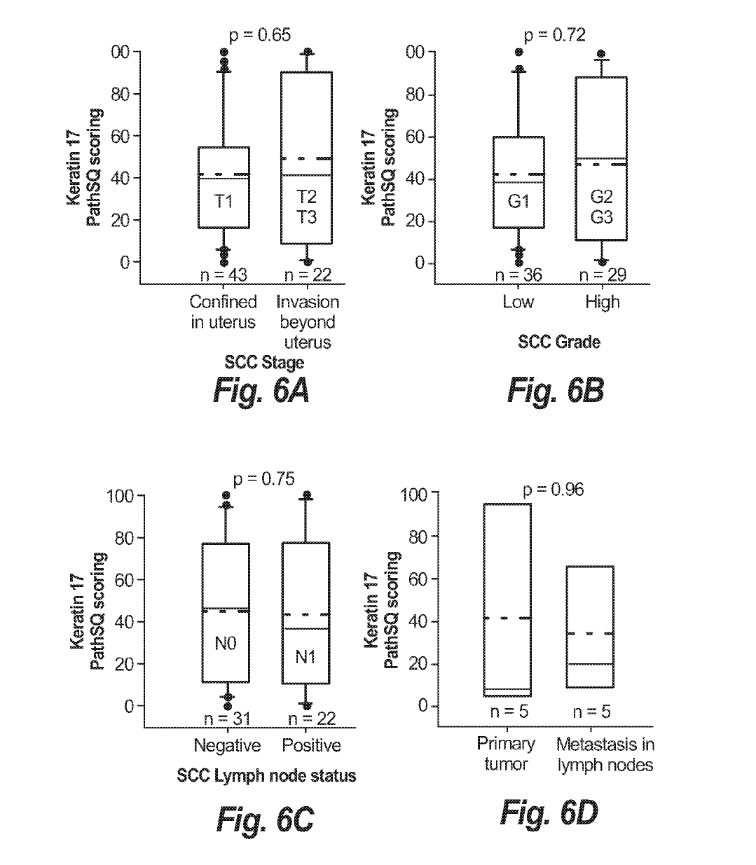

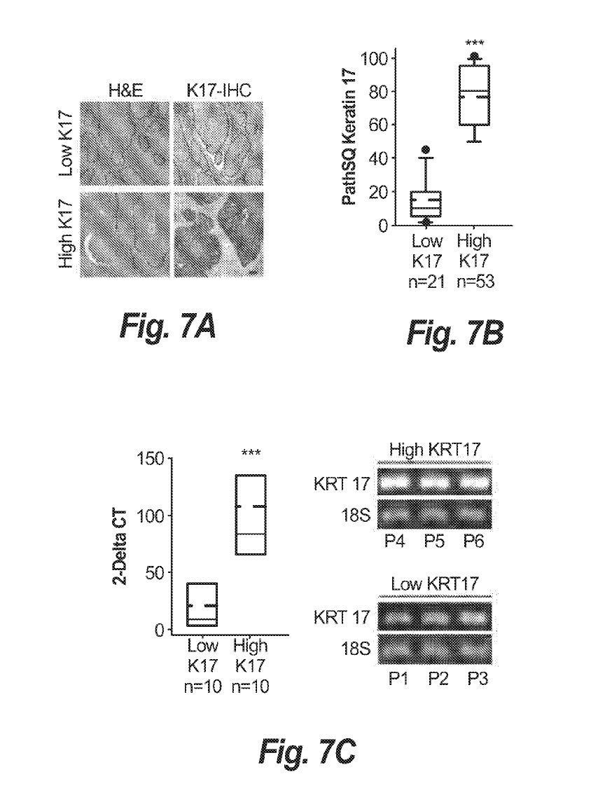

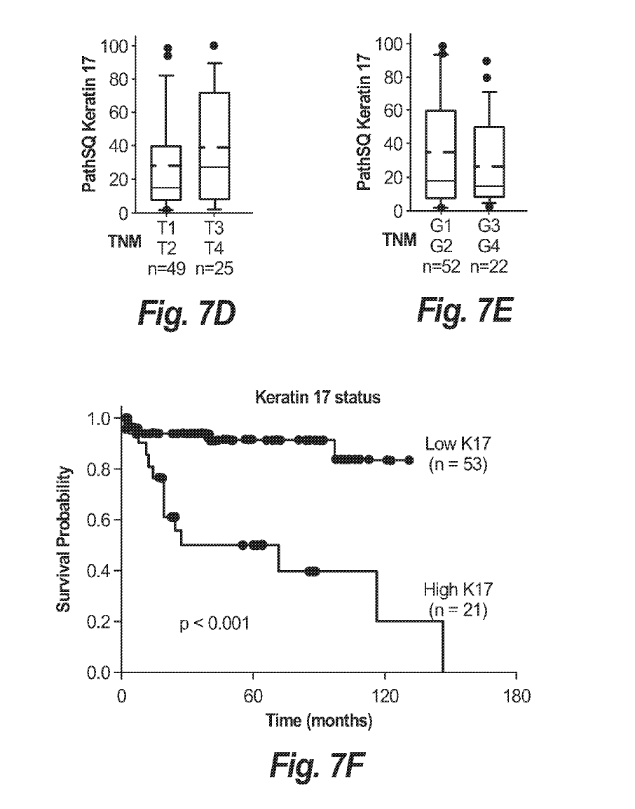

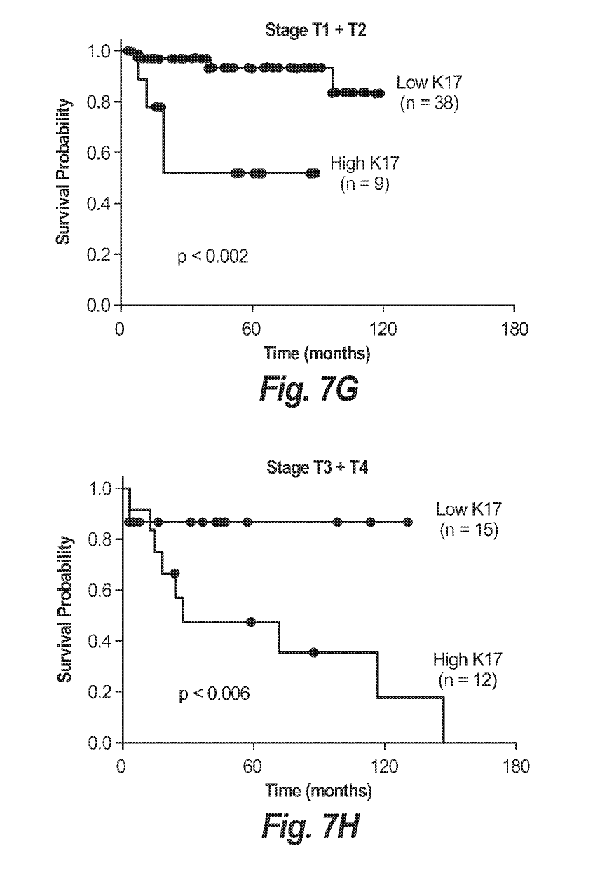

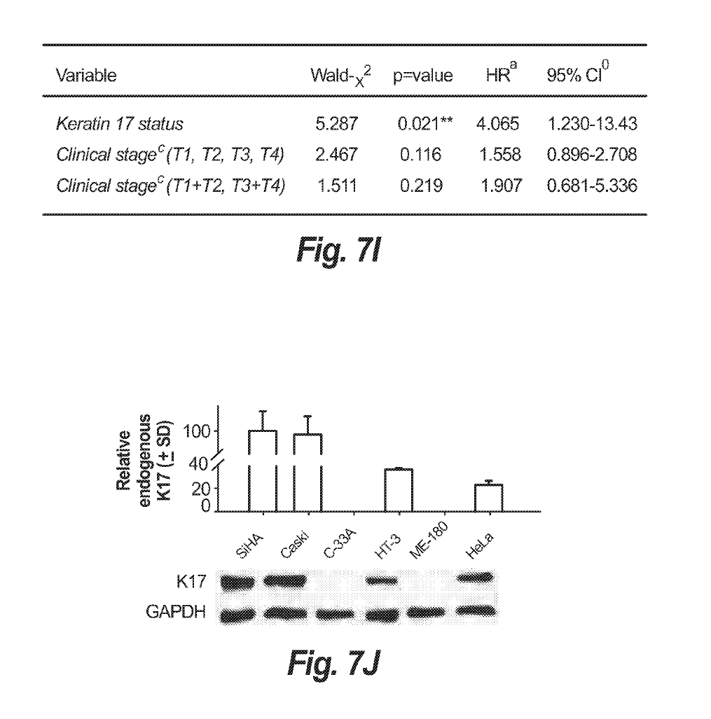

Five-year survival rates of squamous cell carcinoma patients with low KRT17 expression were estimated at 96.97% (95% CI: 80.37-99.57%). Conversely, five-year survival rates of squamous cell carcinoma patients with high KRT17 expression were estimated at 64.31% (95% CI: 39.2-81.21%). A similar trend was observed at the 10-year survival rates of squamous cell carcinoma patients. Ten-year survival rates of squamous cell carcinoma patients with low KRT17 expression were estimated at 96.97% (95% CI: 80.37-99.57%) but ten-year survival rates of squamous cell carcinoma patients with high KRT17 expression were estimated at 52.61% (95% CI: 28.33-72.11%). Although KRT17 expression was associated with overall patient survival, KRT17 expression was not significantly related to tumor stage, histological grade or lymph node status (FIGS. 6-7). Collectively, the data provided herein show that high KRT17 expression is associated with poor overall survival of squamous cell carcinoma patients (Hazard ratio=14.76, 95% CI 1.87-116.58, p=0.01, FIG. 5).

To further validate the use of KRT17 as a prognostic biomarker for patient survival and/or treatment outcome an additional 74 formalin-fixed paraffin-embedded surgical tissue blocks that were retrospectively selected from the archival collections of the UMass Memorial Medical Center, in compliance with IRB-approved protocols at Stony Brook Medicine. The criteria for selection were (i) cases with pathology diagnosis of primary squamous cell carcinoma of the cervix (SCC) and (ii) age of patients older than 18 years at time of diagnosis. Patients with a diagnosis of cancer at other anatomic sites were excluded from the study. SCCs were classified by clinical stage and tumor grade. Survival data were obtained from UMass Memorial Cancer Registry.

Categorical data are described using frequencies and percentages. Continuous data are described using means±standard deviation or standard error. Statistical significance between the means of two groups was determined using Student's t tests or Mann-Whitney U tests. Statistical comparisons of the means of multiple groups were determined using one-way ANOVA or Kruskal-Wallis ANOVA by ranks. Overall survival analyses were performed to validate the relationship between the expression level of keratin 17 and clinical outcomes. The survival curves shown in FIG. 7 were generated using the Kaplan-Meier method. The distribution of the survival functions for keratin 17 expression groups was tested using the log-rank test. Keratin 17 expression groups were tested as defined above, to examine any differences in overall survival rates between the low keratin 17 patients (PathSQ<50) and high keratin 17 (PathSQ≧50) cutoff groups. Multivariate analyses were performed by using the Cox proportional hazards model. This model further examines any differences in the overall survival rates while adjusting for potential confounders deemed to be key prognostic determinants for overall survival such as stage of the cancer. All analyses were performed using SAS 9.3 (SAS Institute, Inc., Cary, N.C., USA) and SigmaPlot 11 (Systat Software, San Jose, Calif., USA). For the statistical significance was set at P<0.05 (α) with power (1−β) at ≧0.8.

TABLE 1
Demographic and clinical characteristics of cases.

| | Biomarker | Diagnostic | Survival |
|---|---|---|---|
| | discovery | validation | analysis |
| | (n = 22) | (n = 102) | (n = 65) |
| Age at diagnosis | | | |
| x̅ (Min-Max) | 37 (19-60) | 39 (19-78) | 51 (28-78) |
| Histology | | | |
| Diagnostic category | | | |
| Normal cervical mucosa | Total | 25 | |
| LSIL^{a} | of | 25 | |
| HSIL^{b} | 22 | 27 | |
| SCC^{c} | | 25 | 65 |
| Clinical stage | | | |
| TI | | | 43 |
| TII | | | 4 |
| TIII | | | 18 |
| Tumor grade | | | |
| Low grade- G1 | | | 36 |
| High grade- G2 and G3 | | | 29 |
| Lymph node status | | | |
| Negative- N0 | | | 31 |
| Positive- N1 | | | 25 |
| Not assessed- NX | | | 9 |

TABLE 2
Keratin 4 and 17 receiver operating curves curve analysis and misclassification
rate results between different diagnostic categories according to PathSQ score.

| | | | AUC^{a} | Sensitivity | Specificity | PPV^{c} | NPV^{d} | Error rate |
|---|---|---|---|---|---|---|---|---|
| Marker | Grouping | Score | (95% CI^{b}) | (95% CI) | (95% CI) | (95% CI) | (95% CI) | (95% CI) |
| KRT4 | SCC^{e} | PathSQ | 66 | 68 | 61 | 36 | 85 | 37 |
| | (n = 25) | | (55-77) | (46-85) | (49-72) | (23-52) | (72-93) | (27-47) |
| | vs other | | | | | | | |
| | (n = 77) | | | | | | | |
| KRT17 | HSIL^{f} + SCC | PathSQ | 96 | 94 | 86 | 87 | 93 | 9 |
| | (n = 52) | | (92-99) | (83-98) | (73-94) | (75-94) | (82-98) | (4-17) |
| | vs Normal + | | | | | | | |
| | LSIL^{g} | | | | | | | |
| | (n = 50) | | | | | | | |

1. A method of identifying a mammalian subject with cervical cancer comprising obtaining a sample from a subject, and detecting KRT4 and/or KRT17 expression in the processed sample obtained from the subject, wherein a reduced level of KRT4 expression or an increased level of KRT17 expression in the processed sample identifies the subject as having cervical cancer.

2. The method of claim 1, further comprising processing the sample.

3. The method of claim 2, wherein said processing the sample comprises dissecting the sample to isolate cells, lysing the isolated cells in a lysis solution comprising urea, isolating the proteins from the lysis solution, digesting the isolated proteins in a digestion solution comprising trypsin and subjecting the resulting mixture to centrifugation to the peptides.

4. The method of claim 1, wherein said sample is selected from the group consisting of: whole blood, tissue, lymph node, or a combination thereof.

5. The method of claim 4, wherein said sample is a tumor biopsy sample or formalin-fixed paraffin-embedded tissue sample.

6. The method of claim 1, wherein the level of KRT4 and/or KRT17 expression in the sample is determined by a process selected from the group consisting of: individual tumor biopsy specimens or tissue microarrays with immunohistochemistry, immunofluorescent assay, Western blotting, or ELISA.

7. The method of claim 1, wherein the level of KRT4 and/or KRT17 expression is measured based on detecting the level of KRT4 or KRT17 mRNA.

8. The method of claim 1, wherein said cervical cancer is squamous cell carcinoma.

9. The method of claim 1, wherein said reduced level of KRT4 expression and/or said increased level of KRT17 expression is determined based on comparing the level of KRT4 or KRT17 expression in the sample to a control level.

10. The method of claim 9, wherein the control level is established from healthy tissue of the subject, or from healthy or cancerous tissue from other subjects.

11. The methods of claim 10, wherein said healthy tissue is squamous mucosa.

12. The method of claim 1, wherein said increased level of KRT17 expression is indicated by the presence of KRT17 expression in greater than 50% of the cells in said sample.

13. The method of claim 1, wherein said reduced level of KRT4 expression is indicated by presence of KRT4 expression in less than 10% of the cells in said sample.

14. A kit for identifying a mammalian subject with cervical cancer comprising instructions describing a method for use according to claim 1.

15. A method of determining the likelihood of survival of a subject having cervical cancer comprising detecting the level of KRT17 expression in a sample obtained from the subject, wherein an increased level of KRT17 expression in the sample identifies the subject as having reduced likelihood of survival.

16. The method of claim 15, wherein said increased level of KRT17 expression is determined by immunohistochemical staining of said sample.

17. The method of claim 16, further comprising comparing the level of KRT17 expression determined by immunohistochemical staining of the sample to KRT17 expression levels in cancerous tissue samples obtained from other subjects with known cervical cancer survival times.

18. The method of claim 16, wherein said increased level of KRT17 expression is indicated by the presence of KRT17 expression in greater than 50% of the cells in said sample.

19. The method of claim 18, wherein said increased level of KRT17 expression is indicated by the presence of KRT17 expression in greater than 52% of the cells in said sample.

20. The method of claim 15, wherein said increased level of KRT17 expression in the sample identifies the subject as having reduced likelihood of survival beyond 50 months from the date of positive diagnosis of cancer.

21. The method of claim 15, wherein said increased level of KRT17 expression in the sample identifies the subject as having reduced likelihood of survival beyond 120 months from the date of positive diagnosis of cancer.

22. A kit for determining the likelihood of survival of a subject having cervical cancer comprising instructions describing a method for use according to claim 15.


SEQUENCE LISTING (7 sequences)

SEQ ID NO: 1 - DNA, 19 bp, Artificial Sequence
  Short interfering RNA molecule against human KRT17 mRNA transcript
agaaagaaccggugaccac

SEQ ID NO: 2 - DNA, 19 bp, Artificial Sequence
  Short interfering RNA molecule against human KRT17 mRNA transcript
cgucaggugcguaccauug

SEQ ID NO: 3 - DNA, 19 bp, Artificial Sequence
  Short interfering RNA molecule against human KRT17 mRNA transcript
gguccaggauggcaagguc

SEQ ID NO: 4 - DNA, 19 bp, Artificial Sequence
  Short interfering RNA molecule against human KRT17 mRNA transcript
ggagaggaugcccaccuga

SEQ ID NO: 5 - DNA, 19 bp, Artificial Sequence
  Short hairpin RNA molecule for knockdown of human KRT17 expression
tcttgtactgagtcaggtg

SEQ ID NO: 6 - DNA, 19 bp, Artificial Sequence
  Short hairpin RNA molecule for knockdown of human KRT17 expression
tctttcttgtactgagtca

SEQ ID NO: 7 - DNA, 19 bp, Artificial Sequence
  Short hairpin RNA molecule for knockdown of human KRT17 expression
ctgtctcaaacttggtgcg